(19) United States
(12) Patent Application Publication
(10) Pub. No.: US 2026/0335248 A1
(43) Pub. Date: Oct. 29, 2026

(54) ANTIFUNGAL AGENT ENCAPSULATED IN A LIPID NANOCRYSTAL FOR TREATING MUCORMYCOSIS

(71) Applicant: MATINAS BIOPHARMA NANOTECHNOLOGIES, INC., Bedminster, NJ (US)

(72) Inventors: Raphael J. Mannino, Glen Gardner, NJ (US); Theresa Matkovits, Bedminster, NJ (US)

(21) Appl. No.: 19/103,882

(22) PCT Filed: Aug. 16, 2023
(86) PCT No.: PCT/US2023/030361

§ 371 Date: Feb. 14, 2025

Related U.S. Application Data
(60) Provisional application No. 63/371,543, filed on Aug. 16, 2022.
(60) Provisional application No. 63/416,045, filed on Oct. 14, 2022.
(60) Provisional application No. 63/447,704, filed on Feb. 23, 2023.

(51) Int. Cl. A61K 9/51; A61K 31/7048; A61K 47/24; A61P 31/10

(52) U.S. Cl. CPC A61K 9/5123; A61K 31/7048; A61K 47/24; A61P 31/10

(57) ABSTRACT

This application relates generally to methods of treating or preventing mucormycosis in a subject in need thereof using, inter alia, encochleated oral formulations of amphotericin B.

### CROSS REFERENCE TO RELATED APPLICATIONS

This application claims the benefit of and relies on the filing date of U.S. Provisional Application No. 63/371,543, filed 16 Aug. 2022; U.S. Provisional Application No. 63/416,045, filed on 14 Oct. 2022; and U.S. Provisional Application No. 63/447,704 filed on 23 Feb. 2023, the entire disclosure of each application is incorporated herein by reference.

### FIELD

This application relates generally to methods of treating or preventing mucormycosis in a subject in need thereof using, inter alia, oral formulations of amphotericin B.

### BACKGROUND

Mucormycosis is a serious, life-threatening infection caused by fungi of the class Zygomycetes, order Mucorales. Fungi belonging to the order Mucorales are distributed into six families, all of which can cause mucormycosis. However, fungi belonging to the family Mucoraceae, and specifically Rhizopus spp., are by far the most common cause of infection. Increasing cases of mucormycosis have been also reported due to infection with Cunninghamella spp. in the Cunninghamellaceae family. The remaining four families of the Mucorales order are less frequent causes of disease.

Mucormycosis is spread by spores of molds, most often through inhalation, contaminated food, or contamination of open wounds. These fungi are common in soils, decomposing organic matter, such as rotting fruit and vegetables, and animal manure, but usually do not affect people. The major risk factors include uncontrolled diabetes mellitus that results in hyperglycemia and ketoacidosis (DKA), other forms of acidosis, treatment with corticosteroids, solid organ or bone marrow transplantation, neutropenia, trauma and burns, malignant haematological disorders, and deferoxamine therapy in patients receiving haemodialysis.

The most common forms of mucormycosis, based on anatomical site, are rhino-orbital/cerebral, pulmonary, cutaneous, gastrointestinal and disseminated. Rhino-orbital/cerebral mucormycosis is found almost exclusively in DKA patients while pulmonary disease is mainly found in neutropenic patients. Cutaneous necrotizing mucormycosis outbreaks in healthy individuals have also been reported and often follow natural disasters or severe trauma. Treatment of mucormycosis generally involves a combination of antifungal drugs, surgically removing infecting tissue and correcting underlying medical problems, such as diabetic ketoacidosis.

Amphotericin B is one of the few antifungal agents approved for the treatment of invasive mucormycosis. Amphotericin B is currently administered intravenously, which requires hospitalization, co-administration of IV fluids and supplemental electrolytes. Moreover, intravenously administered amphotericin B can be toxic, which necessitates rapid and reliable laboratory monitoring. Thus, there exists a need for more effective and less toxic antifungal therapy for treating mucormycosis.

### SUMMARY

Disclosed herein are methods for treating or preventing mucormycosis in a subject in need thereof, which provide surprising benefits in comparison to other methods for treating such diseases. For instance, the methods disclosed herein, which comprise administration of lipid nanocrystal formulations of antifungals, such as amphotericin B, are more effective and less toxic than those using non-encochleated antifungals, such as amphotericin B. Lipid nanocrystal formulations of antifungals, such as amphotericin B, also make it possible to administer the antifungals, such as amphotericin B, orally instead of intravenously, which in turn eliminates the need for hospitalization and close monitoring of patients. Moreover, without the toxicities traditionally associated with intravenously administered amphotericin B, longer courses may be tolerated, which could decrease the incidence of relapse disease.

Accordingly, provided herein are methods of treating or preventing mucormycosis in a subject in need thereof, particularly an immunosuppressed human, comprising administering to the subject a therapeutically effective amount of a lipid nanocrystal comprising an antifungal agent, such as amphotericin B, wherein the lipid nanocrystal comprises a multivalent cation, such as Ca⁺⁺, the antifungal agent, such as amphotericin B, and a lipid component comprising a negatively charged phospholipid, such as phosphatidylserine. In some embodiments, the lipid nanocrystal comprising the antifungal agent, such as amphotericin B, is administered orally to the subject in form of a pharmaceutical composition.

In some aspects, the disclosure provides a method of treating mucormycosis in a subject in need thereof, the method comprising administering to the subject a composition comprising an antifungal agent encapsulated in a lipid nanocrystal, thereby treating mucormycosis in the subject.

In other aspects, the disclosure provides a method of preventing mucormycosis in a subject at risk of infection, the method comprising administering to the subject a composition comprising an antifungal agent encapsulated in a lipid nanocrystal, thereby preventing mucormycosis in the subject.

In some aspects, the disclosure provides use of a composition for treating mucormycosis in a subject, wherein the composition comprises an antifungal agent encapsulated in a lipid nanocrystal.

In other aspects, the disclosure provides use of a composition for preventing mucormycosis in a subject, wherein the composition comprises an antifungal agent encapsulated in a lipid nanocrystal.

In some embodiments, the mucormycosis is one or more of rhinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis. In some embodiments, the mucormycosis is associated with an infectious agent within the order Mucorales. In some embodiments, the infectious agent is a Rhizopus species or a Mucor species. In some embodiments, the infectious agent is selected from Rhizopus oryzae (Rhizopus arrhizus), Rhizopus delemar, Rhizopus microsporus var. rhizopodiformis, Mucor circinelloides, Absidia corymbifera, Apophysomyces elegans, Rhizomucor pusillus and Cunninghamella spp.

In some embodiments, administering comprising oral administration. In some embodiments, the composition is administered to the subject 1-4 times a day for a time period. In some embodiments, the composition is administered once a day for a time period. In some embodiments, the time period is at least 7 days.

In some embodiments, the composition comprises 5-30 mg/mL of the antifungal agent. In some embodiments, 100-2,000 mg/day of the antifungal agent is administered to the subject.

In some embodiments, treating comprises reducing fungal burden in the subject. In some embodiments, fungal burden is reduced in the brain and/or lungs of the subject. In some embodiments, the fungal burden is reduced by at least 10%, at least 20%, at least 30%, at least 40%, at least 50%, at least 60%, at least 70%, at least 80%, at least 90%, at least 95%, or at least 99% relative to fungal burden prior to administration of the composition.

In some embodiments, the subject is immunosuppressed. In some embodiments, the subject has an HIV infection and/or cancer. In some embodiments, the subject is a human subject. In some embodiments, the antifungal agent is selected from the group: amphotericin B, amphotec, posaconazole, isavuconazole, voricoazole, fluconazole, itraconazole, caspofungin acetate, micafungin, and combinations thereof. In some embodiments, the antifungal agent is amphotericin B.

In some embodiments, the lipid nanocrystal comprises a multivalent cation and a lipid component comprising a negatively charged phospholipid. In some embodiments, the negatively charged phospholipid is phosphatidylserine. In some embodiments, the phosphatidylserine is soy phosphatidylserine. In some embodiments, the lipid component comprises a neutrally charged lipid. In some embodiments, the neutrally charged lipid is phosphatidylcholine. In some embodiments, the multivalent cation is Ca⁺⁺, Zn⁺⁺, Ba⁺⁺, or Mg⁺⁺. In some embodiments, the multivalent cation is Ca⁺⁺. In some embodiments, the lipid component comprises phosphatidylserine and phosphatidylcholine, and wherein the multivalent cation is Ca⁺⁺.

In some embodiments, the MIC₅₀ (μg/mL) of the antifungal agent is less than 0.005. In some embodiments, the MIC₅₀ (μg/mL) of the antifungal agent is less than 0.01.

In some embodiments, the composition comprises a pharmaceutically acceptable carrier. In some embodiments, the composition comprises purified water, EDTA, vitamin E, calcium chloride, methylparaben sodium, propylparaben sodium, sodium hydroxide, dehydrated alcohol, monobasic potassium phosphate, potassium sorbate, acesulfame potassium, and optionally flavoring.

In some aspects, the disclosure provides a method of treating mucormycosis in a subject in need thereof, the method comprising administering to the subject a composition comprising amophotericin B encapsulated in a lipid nanocrystal comprising Ca⁺⁺ and a lipid component comprising phosphatidylserine and phosphatidylcholine, thereby treating mucormycosis in the subject. In some embodiments the composition is administered at a dose of 100-2,000 mg amphotericin B per day.

In some aspects, the disclosure provides a kit comprising a composition comprising an antifungal agent encapsulated in a lipid nanocrystal and instructions for administering the composition to a subject in need thereof, wherein the subject comprises mucormycosis or is at risk of contracting mucormycosis. In some embodiments, the antifungal agent is amphotericin B. In some embodiments, the lipid nanocrystal comprises a multivalent cation and a lipid component comprising a negatively charged phospholipid, optionally wherein the multivalent cation is Ca⁺⁺ and the negatively charged phospholipid is phosphatidylserine, further optionally wherein the lipid component comprises a neutrally charged lipid, optionally wherein the neutrally charged lipid is phosphatidylcholine.

### BRIEF DESCRIPTION OF THE DRAWING

The accompanying drawings, which are incorporated in and constitute a part of this specification, illustrate certain embodiments, and together with the written description, serve to explain certain principles of the methods and compositions disclosed herein.

FIG. 1A provides a graph showing survival of neutropenic mice (n=10/group) infected with 1.8×10⁴ spores of R. delemar (EXP-1; Table 1) and treated with i) placebo; ii) 5 mg/kg of MAT2203 (qd, i.e., once daily); iii) 15 mg/kg of MAT2203 (qd); iv) 45 mg/kg of MAT2203 (qd); or, v) 10 mg/kg LAMB (qd). Treatment was initiated 16 hours post infection with R. delemar and continued for 7 days for MAT2203 and 4 days for LAMB. MAT2203 and LAMB were administered by oral and intravenous administration, respectively. P values are determined by Log Rank test relative to placebo-treated mice.

FIG. 1B provides a graph showing survival of neutropenic mice (n=10/group) infected with 1.5×10⁴ spores of R. delemar (EXP-2; Table 2) and treated with i) placebo; ii) 7.5 mg/kg of MAT2203 (bid, i.e., twice daily); iii) 15 mg/kg of MAT2203 (bid); iv) 15 mg/kg of MAT2203 (qd); or v) 10 mg/kg LAMB (qd). Treatment was initiated 16 hours post infection and continued for 7 days for MAT2203 and 4 days for LAMB. MAT2203 and LAMB were administered by oral and intravenous administration, respectively. P values are determined by Log Rank test relative to placebo-treated mice.

FIG. 2 shows combined data of survival of neutropenic mice infected with R. delemar and treated with MAT2203 or LAMB, as described in Example 1 and FIGS. 1A-1B. P values on each of graphs are versus placebo-treated mice. Data in the table include the median survival times and the overall survival by day 21 post infection.

FIGS. 3A-3B shows reduction in lung tissue (FIG. 3A) and brain tissue (FIG. 3B) fungal burden of immunosuppressed mice infected with R. delemar, as described in Example 1 (Table 3). Mice (n=10/group) infected intratracheally with R. delemar (inhaled inoculum of 2.9×10⁴ spores/mouse) and 16 hours later treated with i) placebo; ii) MAT2203 5 mg/kg (qd); iii) MAT2203 15 mg/kg (qd); or iv) LAMB 10 mg/kg (qd). On day +4 after infection, organs were collected and processed for tissue fungal burden by qPCR. Data=median+interquartile range and the y axis represents the lower limit of detection. Intergroup P values shown as a dark line. Both MAT2203 at 15 mg/kg and LAMB resulted in a statistically significant reduction in lung and brain fungal burden versus placebo control (Wilcoxon Rank sum test).

FIG. 4 shows survival of neutropenic mice (n=10/group) infected with 2.5×10⁶ M. circineloides (Table 4) and treated with i) placebo; ii) 15 mg/kg of MAT2203 (qd); or, iii) 10 mg/kg LAMB (qd). MAT2203 and LAMB were administered by oral and intravenous administration, respectively. P values are determined by Log Rank test relative to placebo-treated mice. Data in the table include the median survival times and the overall survival by day 21 post infection.

FIGS. SA-SB shows reduction in lung tissue (FIG. 5A) and brain tissue (FIG. 5B) fungal burden of neutropenic mice infected with M. circineloides, as described in Example 1 (Table 5). Mice (n=10/group) infected intratracheally with 2.9×10⁴ spores/mouse and 16 hours later treated with i) placebo, ii) MAT2203 15 mg/kg (qd); or, iii) with LAMB 10 mg/kg (qd). On day +4 organs were collected and processed for tissue fungal burden by qPCR. Data=median+interquartile range and the y axis represents the lower limit of detection. Intergroup P values shown as a dark line. Both MAT2203 at 15 mg/kg and LAMB resulted in a statistically significant reduction in lung and brain fungal burden versus placebo control (Wilcoxon Rank sum test).

FIG. 6 shows histological examination of lung sections taken from mice infected with M. circineloides and stained with GMS revealed fungal pneumonia (indicated by the abscesses in the placebo mice with broad aseptate hyphae), as described in Example 1. While evidence of pneumonia still existed in mice treated with either MAT2203 or LAMB, the number of fungal abscesses were less with shorter and damaged fungal hyphae.

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the disclosure, and should not be interpreted as limiting the scope of the disclosure.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of +20%, +10%, +5%, +1%, +0.9%, +0.8%, #0.7%, +0.6%, +0.5%, +0.4%, +0.3%, +0.2% or +0.1% from the specified value as such variations are appropriate to perform the disclosed methods and/or to make and use the disclosed compositions. When “about” is present before a series of numbers or a range, it is understood that “about” can modify each of the numbers in the series or range.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The terms “at least,” “less than” or “more than” prior to a number or series of numbers (e.g., “at least two”) is understood to include the number adjacent to the term “at least,” “less than” or “more than,” and all subsequent numbers or integers that could logically be included, as clear from context. When the term “at least,” “less than” or “more than” is present before a series of numbers or a range, it is understood that “at least,” “less than” or “more than” can modify each of the numbers in the series or range.

As used herein, the term “in some embodiments,” “in other embodiments,” or the like, refers to embodiments of all aspects of the disclosure, unless the context clearly indicates otherwise.

The term “mucormycosis,” as used herein, refers to a fungal condition caused by fungi of the order Mucorales. Mucormycosis is a life-threatening fungal infection almost uniformly affecting immunocompromised hosts in either developing or industrialized countries. Fungi belonging to the order Mucorales are distributed into at least six families, all of which can cause cutaneous and deep infections. Species belonging to the family Mucoraceae are isolated more frequently from patients with mucormycosis than any other family. Among the Mucoraceae, Rhizopus oryzae (Rhizopus arrhizus) is a common cause of infection. Other exemplary species of the Mucoraceae family that cause a similar spectrum of infections include, for example, Rhizopus microsporus var. rhizopodiformis, Absidia corymbifera, Apophysomyces elegans, Mucor species, Rhizomucor pusillus and Cunninghamella spp (Cunninghamellaceae family). Mucormycosis is well known in the art and includes, for example, rhinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis.

The term “subject” is used interchangeably herein with the terms “patient” and “individual” to refer to any subject for whom diagnosis or therapy is desired, particularly mammals, such as humans. In some embodiments, the subject is a human. Because mucormycosis affects almost uniformly immunocompromised hosts, in some embodiments, the subject is immunosuppressed.

The term “in need thereof” means that the subject has been identified or suspected as having a need for the particular method or treatment. In some embodiments, the identification can be by any means of diagnosis or observation. In any of the methods described herein, the subject can be in need thereof. In some embodiments, the subject in need thereof is a human suspected of having mucormycosis. In some embodiments, the subject in need thereof is a human diagnosed with mucormycosis. In some embodiments, the subject in need thereof is a human seeking treatment for mucormycosis. In some embodiments, the subject in need thereof is a human undergoing treatment for mucormycosis.

The term “therapeutically effective amount” refers to a dosage or amount that is therapeutically sufficient for treating an indicated disease or condition, such as mucormycosis. In some embodiments, a “therapeutically effective amount” is an amount sufficient to prevent, reduce or attenuate mucormycosis in the subject or patient.

The terms “treatment” or “treating” and the like refer to any treatment of any disease or condition in an animal, such as a bird or mammal, e.g. particularly a human or a mouse, and includes inhibiting a disease, condition, or symptom of a disease or condition, e.g., arresting its development and/or delaying its onset or manifestation in the patient or relieving a disease, condition, or symptom of a disease or condition, e.g., causing regression of the condition or disease and/or its symptoms.

The term “preventing” or “prevention,” as it is used herein is intended to mean a prevention of a clinical symptom indicative of mucormycosis. Such prevention can include, for example, the maintenance of normal physiological indicators in an individual at risk of infection by a fungus or fungi prior to the development of overt symptoms of the condition or prior to diagnosis of the condition. Therefore, the term “preventing” includes the prophylactic treatment of individuals to guard them from the occurrence of mucormycosis.

“Lipid nanocrystal” or “Lipid nanocrystals,” as used herein, refer to anhydrous, stable, multi-layered lipid crystals that spontaneously form upon the interaction of negatively charged lipids, such as phospholipids, and a multivalent cation, such as calcium (see, for example, U.S. Pat. Nos. 4,078,052; 5,643,574; 5,840,707; 5,994,318; 6,153,217; 6,592,894, as well as PCT Publ. Nos. WO 2004/091572; WO 2004/091578; WO 2005/110361, WO 2012/151517, and WO2014/022414, and U.S. Pat. Publ. 2010/0178325; each of which is incorporated fully herein by reference). These lipid nanocrystals are also referred to in the art as cochleates and these terms are used interchangeably in this application. A lipid nanocrystal or cochleate has a unique multilayered structure consisting of a large, continuous, solid, lipid bilayer sheet or strata rolled up in a spiral or as stacked sheets with no internal aqueous space. This unique structure provides protection from degradation for associated, encapsulated or “encochleated” molecules. Divalent cation concentrations in vivo in serum and mucosal secretions are such that the lipid nanocrystal structure is maintained. Hence, the majority of lipid nanocrystal-associated molecules are present in the inner layers of a solid, stable, impermeable structure. Once within the interior of a cell, however, the low calcium concentration results in the opening of the lipid nanocrystal and release of the molecule that had been formulated into the lipid nanocrystals. Accordingly, lipid nanocrystal formulations remain intact in physiological fluids, including mucosal secretions, plasma and gastrointestinal fluid, thereby mediating the delivery of biologically active compounds by many routes of administration, including mucosal, intravenous and oral.

The lipid nanocrystals used in the methods of the disclosure comprise at least the following components: a multivalent cation, an antifungal agent, and a lipid component comprising a negatively charged phospholipid. In some embodiments, the antifungal agent is in an aqueous medium and encapsulated in the lipid nanocrystal.

The term “multivalent cation” refers to a divalent cation or higher valency cation, or any compound that has at least two positive charges, including mineral cations such as calcium, barium, zinc, iron and magnesium and other elements capable of forming ions or other structures having multiple positive charges capable of chelating and bridging negatively charged lipids. Typically, a multivalent compound is used to precipitate the lipid nanocrystals of the disclosure from a liposome solution as described in, for example, U.S. Pat. Nos. 4,078,052; 5,643,574; 5,840,707; 5,994,318; 6,153,217; 6,592,894, as well as PCT Publ. Nos. WO 2004/091572; WO 2004/091578; WO 2005/110361, WO 2012/151517, and WO2014/022414, and U.S. Pat. Publ. 2010/0178325; each of which is incorporated fully herein by reference. Any multivalent compound may be used; however, typically, such compounds are divalent cations such as Ca⁺⁺, Zn⁺⁺, Ba⁺⁺, and Mg⁺⁺. In some embodiments, sources of these cations include the chloride salts of calcium, zinc, barium, and magnesium. In some embodiments, CaCl₂) is a source of divalent cations. Depending on the multivalent compound used for the precipitation, the multivalent cation comprised in the lipid nanocrystals disclosed herein may vary. In some embodiments, the multivalent cation comprised in the lipid nanocrystals disclosed herein is Ca⁺⁺. In some embodiments, the multivalent cation is Zn⁺⁺. In some embodiments, the multivalent cation is Ba⁺⁺. In some embodiments, the multivalent cation is Mg⁺⁺.

“Negatively charged phospholipid,” as used herein, refers to a phospholipid that has a net negative charge at physiological pH. The negatively charged phospholipid may comprise a single type of negatively charged phospholipid, or a mixture of two or more different, negatively charged, phospholipids. In some embodiments, the lipid component of the lipid nanocrystals according to the disclosure comprises a single type of negatively charged phospholipid. In other embodiments, the lipid component of the lipid nanocrystals comprises a mixture of two or more different, negatively charged, phospholipids.

### Methods of Treating Mucormycosis

The present disclosure is directed to a method of treating or preventing mucormycosis in a subject in need thereof, the method comprising administering to the subject a therapeutically effective amount of a lipid nanocrystal comprising an antifungal agent, wherein the lipid nanocrystal comprises a multivalent cation, the antifungal agent, and a lipid component comprising a negatively charged phospholipid. In some embodiments, the methods of the disclosure treat mucormycosis in the subject. In other embodiments, the methods of the disclosure prevent mucormycosis in the subject. In some embodiments, the mucormycosis treated or prevented by the method disclosed herein is associated with an infectious agent within the order Mucorales. Fungi belonging to the order Mucorales are currently distributed into the families of Choanephoraceae, Cunninghamellaceae, Mucoraceae, Mycotyphaceae, Phycomycetaceae, Pilobolaceae, Saksenacaceae, Syncephalastraceae, and Umbelopsidaceae. Each of these fungi families consists of one or more genera. For example, fungi belonging to the order Mucorales, family Mucoraceae, are further classified into the genera of Absidia (e.g., A. corymbifera), Actinomucor (e.g., A. elegans), Amylomyces (e.g., A. rouxii), Apophysomyces, Backusella (e.g., B. circina), Benjaminiella (e.g., B. multispora), Chaetocladium (e.g., C. brefeldii), Circinella (e.g., C. angarensis), Cokeromyces (e.g., C. recurvatus), Dicranophora (e.g., D. fulva), Ellisomyces (e.g., E. anomalus), Helicostylum (e.g., H. elegans), yphomucor (e.g., H. assamensis), Kirkomyces (e.g., K. cordensis), Mucor (e.g., M. amphibiorum), Parasitella (e.g., P. parasitica), Philophora (e.g., P. agaricina), Pilaira (e.g., P. anomala), Pirella (e.g., P. circinans), Rhizomucor (e.g., R. endophyticus), Rhizopodopsis (e.g., R. javensis), Rhizopus, Sporodiniella (e.g., S. umbellata), Syzygites (e.g., S. megalocarpus), Thamnidium (e.g., T. elegans), Thermomucor (e.g., T. indicae-seudaticae), and Zygorhynchus (e.g., Z. californiensis). The genus Rhizopus, for example, consists of R. azygosporus, R. caespitosus, R. homothallicus, R. oryzae, and R. schipperae species. In some embodiments, the infectious agent associated with the mucormycosis is a Rhizopus species or a Mucor species. In some embodiments, the infectious agent associated with the mucormycosis is selected from Rhizopus oryzae (Rhizopus arrhizus), Rhizopus delemar, Rhizopus microsporus var. rhizopodiformis, Mucor circinelloides, Absidia corymbifera, Apophysomyces elegans, Rhizomucor pusillus and Cunninghamella spp (Cunninghamellaceae family).

In some embodiments, the fungal infection is superficial. In some embodiments, the fungal infection is locally invasive. In some embodiments, the fungal infection is disseminated.

Accordingly, in some embodiments, the mucormycosis treated or prevented by the method disclosed herein comprises rhinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis. In some embodiments, the mucormycosis treated or prevented by the method disclosed herein comprises pulmonary mucormycosis. In some embodiments, the mucormycosis treated or prevented by the method disclosed herein comprises rhinocerebral mucormycosis. In some embodiments, the mucormycosis treated or prevented by the method disclosed herein comprises gastrointestinal mucormycosis. In some embodiments, the mucormycosis treated or prevented by the method disclosed herein comprises disseminated mucormycosis. In some embodiments, the mucormycosis treated or prevented by the method disclosed herein comprises bone mucormycosis. In some embodiments, the mucormycosis treated or prevented by the method disclosed herein comprises mediastinum mucormycosis. In some embodiments, the mucormycosis treated or prevented by the method disclosed herein comprises trachea mucormycosis. In some embodiments, the mucormycosis treated or prevented by the method disclosed herein comprises kidney mucormycosis. In some embodiments, the mucormycosis treated or prevented by the method disclosed herein comprises peritoneum mucormycosis. In some embodiments, the mucormycosis treated or prevented by the method disclosed herein comprises superior vena cava mucormycosis. In some embodiments, the mucormycosis treated or prevented by the method disclosed herein comprises external otitis mucormycosis.

In some embodiments, the methods described herein reduce fungal burden in one or more tissues of a subject. In some embodiments, the methods described herein reduce fungal burden in one or more of lung, brain, central nervous system tissue, gastrointestinal tissue, bone mediastinum, trachea, kidney, peritoneum, superior vena cava, or external otitis. In some embodiments, the methods described herein reduce fungal burden in one or more of lung, brain, central nervous system tissue, gastrointestinal tissue, bone mediastinum, trachea, kidney, peritoneum, superior vena cava, or external otitis of a subject by at least 10%, at least 20%, at least 30%, at least 40%, at least 50%, at least 60%, at least 70%, at least 80%, at least 90%, at least 95%, or at least 99% relative to fungal burden prior to administration of a lipid nanocrystal described herein.

Lungs and brains are the primary and secondary target organs in mucormycosis, respectively (Luo et al., Antimicrob. Agents Chemother., 2013, 57 (7): 3340-3347). Thus, the effectiveness in reducing lungs and/or brain fungal burdens can be a way to determine the effectiveness of a given treatment for mucormycosis. For instance, it has been reported that liposomal amphotericin B or Posaconazole treatments were effective in reducing the fungal burdens in lungs and brain compared with placebo (Luo et al., Antimicrob. Agents Chemother., 2013, 57 (7): 3340-3347). Accordingly, in some embodiments, administering the therapeutically effective amount of the lipid nanocrystals of the disclosure, or pharmaceutical compositions thereof, reduces fungal burden in the lung of the subject. In some embodiments, administering the therapeutically effective amount of the lipid nanocrystals of the disclosure, or pharmaceutical compositions thereof, reduces fungal burden in the brain of the subject. In some embodiments, administering the therapeutically effective amount of the lipid nanocrystals of the disclosure, or pharmaceutical compositions thereof, reduces fungal burden in both the lung and brain of the subject.

In some embodiments, the methods described herein reduce fungal burden in the lung of a subject by at least 10%, at least 20%, at least 30%, at least 40%, at least 50%, at least 60%, at least 70%, at least 80%, at least 90%, at least 95%, or at least 99% relative to fungal burden prior to administration of a lipid nanocrystal described herein. In some embodiments, the methods described herein reduce fungal burden in the brain of a subject by at least 10%, at least 20%, at least 30%, at least 40%, at least 50%, at least 60%, at least 70%, at least 80%, at least 90%, at least 95%, or at least 99% relative to fungal burden prior to administration of a lipid nanocrystal described herein. In some embodiments, the methods described herein reduce fungal burden in the lung and brain of a subject by at least 10%, at least 20%, at least 30%, at least 40%, at least 50%, at least 60%, at least 70%, at least 80%, at least 90%, at least 95%, or at least 99% relative to fungal burden prior to administration of a lipid nanocrystal described herein.

In some embodiments, the antifungal agent encapsulated in a lipid nanocrystal is orally administered to a subject and is at least as effective as intravenously administering a liposomal antifungal agent to a subject. In some embodiments, the amphotericin B encapsulated in a lipid nanocrystal is orally administered to a subject and is at least as effective as intravenously administering liposomal amphotericin B to a subject. In some embodiments, the antifungal agent encapsulated in a lipid nanocrystal is orally administered to a subject and is more effective than intravenously administering a liposomal antifungal agent to a subject. In some embodiments, the amphotericin B encapsulated in a lipid nanocrystal is orally administered to a subject and is more effective than intravenously administering liposomal amphotericin B to a subject.

In some embodiments, the antifungal agent encapsulated in a lipid nanocrystal is orally administered to a subject and is at least 10%, at least 20%, at least 30%, at least 40%, at least 50%, at least 60%, at least 70%, at least 80%, at least 90%, at least 95%, or at least 99% as effective as intravenously administering a liposomal antifungal agent to a subject. In some embodiments, the amphotericin B encapsulated in a lipid nanocrystal is orally administered to a subject and is at least 10%, at least 20%, at least 30%, at least 40%, at least 50%, at least 60%, at least 70%, at least 80%, at least 90%, at least 95%, or at least 99% as effective as intravenously administering liposomal amphotericin B to a subject.

In some embodiments, the antifungal agent encapsulated in a lipid nanocrystal reduces fungal burden in a patient to the same or similar level as an intravenously administered liposomal antifungal agent. In some embodiments, the amphotericin B encapsulated in a lipid nanocrystal reduces fungal burden in a patient to the same or similar level as an intravenously administered liposomal amphotericin B. In some embodiments, the antifungal agent encapsulated in a lipid nanocrystal increases the reduction in fungal burden compared to the reduction by an intravenously administered liposomal antifungal agent. In some embodiments, the amphotericin B encapsulated in a lipid nanocrystal increases the reduction in fungal burden compared to the reduction by an intravenously administered liposomal amphotericin B.

In some embodiments, the antifungal agent encapsulated in a lipid nanocrystal is administered to a subject in a therapeutically effective amount. In some embodiments, the therapeutically effective amount of antifungal agent encapsulated in a lipid nanocrystal is less than the standard of care dose of a liposomal amphotericin B. In some embodiments, the therapeutically effective amount of antifungal agent encapsulated in a lipid nanocrystal is less than the standard of care dose of a liposomal amphotericin B, wherein the standard of care dose is about 3 mg/kg/day to about 10 mg/kg/day.

In some embodiments, methods for treating mucormycosis with an antifungal agent encapsulated in a lipid nanocrystal described herein results in improvements of clinical symptoms attributed to the infection. In some embodiments, clinical symptoms attributed to the infection include, for example, general appearance including appearance of the skin, head, eyes, ears, nose, throat, neck, trunk, or lymph nodes, or the respiratory, cardiovascular, gastrointestinal, genitourinary, musculoskeletal, neurological, psychological, lymphatic/hematological, and endocrine/metabolic systems of the mammal. In some embodiments, improvements in one or more outcome measures are by at least 10%, at least 15%, at least 20%, at least 25%, at least 30%, at least 35%, at least 40%, at least 45%, at least 50%, at least 55%, at least 60%, at least 65%, at least 70%, at least 75%, at least 85%, at least 90%, at least 95%, or more than 95%. In some embodiments, the administration of a lipid nanocrystal described herein results in one or more outcome measures improving by at least 0.5 fold, 1 fold, 1.5 fold, 2 fold, 2.5 fold, 3 fold, 3.5 fold, 4 fold, 5 fold, 6 fold, 7 fold, 8 fold, 9 fold, 10 fold, or more than 10 fold. Improvements, in some embodiments, are compared to a control. In some embodiments, a control is an individual who does not receive a lipid nanocrystal. In some embodiments, the control is baseline for the individual prior to receiving the lipid nanocrystal.

Improvements in outcome measures are assessed with repeated assessments taken during treatment with lipid nanocrystal and a comparison against the baseline assessment and/or any prior assessments. Evaluating a subject for fungal infections and assessing efficacy of treatment with the lipid nanocrystal, includes multiple modalities of diagnostic testing, including: radiological assessments including CT scanning of the CNS, chest, sinuses, and abdomen; fungal culture and microscopy of respiratory specimens; blood, serum, or bronchoalveolar fluid fungal antigen testing; blood, serum, or bronchoalveolar fluid pathogenic DNA testing; biopsy of the lung; urine test; and other molecular testing of respiratory samples.

In some embodiments, the methods described herein improve the survival rate of a subject administered an antifungal agent encapsulated in a lipid nanocrystal relative to a subject administered a standard of care treatment. In some embodiments, the methods described herein improve the survival rate of a subject administered an antifungal agent encapsulated in a lipid nanocrystal relative to a subject administered a standard of care treatment by at least 10%, at least 20%, at least 30%, at least 40%, at least 50%, at least 60%, at least 70%, at least 80%, at least 90%, or at least 90%,

### Dosage and Administration

There are currently only a few antifungal agents approved for the treatment of mucormycosis and amphotericin B is one of them. Amphotericin B is usually administered intravenously, which requires hospitalization, co-administration of IV fluids and supplemental electrolytes, and can be toxic. Because the lipid nanocrystals of the disclosure can be administered orally, amphotericin B formulated in form of a lipid nanocrystal according to the present disclosure can be administered orally instead of intravenously to avoid any inconvenience and toxicity associated with intravenous administration. Accordingly, in some embodiments, the antifungal agent comprised in the lipid nanocrystals of the disclosure is amphotericin B and the lipid nanocrystals of the disclosure, or pharmaceutical compositions thereof, are administered to the subject orally. In some embodiments, the orally administering amphotericin B in form of a lipid nanocrystal according to the present disclosure is at least as effective as intravenously administering amphotericin B, such as liposomal amphotericin B. Because orally administering amphotericin B in form of a lipid nanocrystal according to the present disclosure makes it possible to avoid toxicity associated with intravenously administering amphotericin B, such as liposomal amphotericin B, in some embodiments therefore, the antifungal agent comprised in the lipid nanocrystals of the disclosure, such as amphotericin B, is administered at a higher dosage than the standard of care, such as liposomal amphotericin B. Alternatively, in view of the lower MIC₅₀ and MIC₉₀ values observed for MAT2203, as compared to LAMB (see e.g., Example 1), the antifungal agent comprised in the lipid nanocrystals of the disclosure, such as amphotericin B, is administered at a lower dosage than the standard of care, such as liposomal amphotericin B.

The amount of the antifungal agent, such as amphotericin B, which will be effective in the treatment or prevention of mucormycosis can be determined using clinical techniques. In addition, in vitro assays can optionally be employed to help identify optimal dosage ranges. The precise dose to be employed in the formulation will also depend on the route of administration, and the seriousness of the disease or condition, and should be decided according to the judgment of the practitioner and each subject's circumstances. Effective doses can be extrapolated from dose-response curves derived from in vitro or animal model test systems.

Based on the disclosure of this application, one skilled in the art can readily determine an appropriate dosage regimen for administering a therapeutically effective amount of the lipid nanocrystal of the disclosure, or a pharmaceutical composition thereof, to a given subject. For example, in some embodiments, the lipid nanocrystals of the disclosure, or pharmaceutical compositions thereof, can be administered to a subject once daily, twice daily, three times a day, or four times a day. In some embodiments, the antifungal agent encapsulated in a lipid nanocrystal is administered once per day. In some embodiments, the antifungal agent encapsulated in a lipid nanocrystal is administered twice per day. In some embodiments, the antifungal agent encapsulated in a lipid nanocrystal is administered three times per day. In some embodiments, the antifungal agent encapsulated in a lipid nanocrystal is administered four times per day.

In other embodiments, the lipid nanocrystals of the disclosure, or pharmaceutical compositions thereof, can be administered to a subject once weekly, every two weeks, etc. In some embodiments, the antifungal agent encapsulated in a lipid nanocrystal is administered once per week, twice per week, three times per week, or four times per week. In some embodiments, the antifungal agent encapsulated in a lipid nanocrystal is administered once per week. In some embodiments, the antifungal agent encapsulated in a lipid nanocrystal is administered twice per week. In some embodiments, the antifungal agent encapsulated in a lipid nanocrystal is administered three times per week. In some embodiments, the antifungal agent encapsulated in a lipid nanocrystal is administered four times per week. In some embodiments, the antifungal agent encapsulated in a lipid nanocrystal is administered 1-2 times weekly. In some embodiments, the antifungal agent encapsulated in a lipid nanocrystal is administered 1-3 times weekly. In some embodiments, the antifungal agent encapsulated in a lipid nanocrystal is administered 1-4 times weekly. In some embodiments, the antifungal agent encapsulated in a lipid nanocrystal is administered 2-3 times weekly. In some embodiments, the antifungal agent encapsulated in a lipid nanocrystal is administered 2-4 times weekly. In some embodiments, the antifungal agent encapsulated in a lipid nanocrystal is administered 3-4 times weekly.

In some embodiments, the lipid nanocrystals of the disclosure, or pharmaceutical compositions thereof, are administered for a period of from about 3 to about 28 days, or from about 7 to about 10 weeks. In some embodiments, the antifungal agent encapsulated in a lipid nanocrystal, or pharmaceutical compositions thereof, are administered for a period of from about 3 days to about 28 days. In some embodiments, the antifungal agent encapsulated in a lipid nanocrystal or pharmaceutical compositions thereof, are administered for a period of from about 7 weeks to about 10 weeks.

In some embodiments, the lipid nanocrystals of the disclosure, or pharmaceutical compositions thereof, are administered to the subject continuously for at least about 7 days. In some embodiments, the antifungal agent encapsulated in a lipid nanocrystal is administered daily for at least 1, 2, 3, 4, 5, 6, 7, 8, 9, 10, 11, or 12 weeks. In some embodiments, the antifungal agent encapsulated in a lipid nanocrystal is administered daily for at least 1 week. In some embodiments, the antifungal agent encapsulated in a lipid nanocrystal is administered daily for at least 2 weeks. In some embodiments, the antifungal agent encapsulated in a lipid nanocrystal is administered daily for at least 3 weeks. In some embodiments, the antifungal agent encapsulated in a lipid nanocrystal is administered daily for at least 4 weeks. In some embodiments, the antifungal agent encapsulated in a lipid nanocrystal is administered daily for at least 5 weeks. In some embodiments, the antifungal agent encapsulated in a lipid nanocrystal is administered daily for at least 6 weeks. In some embodiments, the lipid nanocrystal formulation is administered daily for at least 7 weeks. In some embodiments, the lipid nanocrystal formulation is administered daily for at least 8 weeks. In some embodiments, the lipid nanocrystal formulation is administered daily for at least 9 weeks. In some embodiments, the antifungal agent encapsulated in a lipid nanocrystal is administered daily for at least 10 weeks. In some embodiments, the antifungal agent encapsulated in a lipid nanocrystal is administered daily for at least 11 weeks. In some embodiments, the antifungal agent encapsulated in a lipid nanocrystal is administered daily for at least 12 weeks. In some embodiments, the antifungal agent encapsulated in a lipid nanocrystal is administered daily for at least 3 months. In some embodiments, the antifungal agent encapsulated in a lipid nanocrystal is administered daily for at least 4 months. In some embodiments, the antifungal agent encapsulated in a lipid nanocrystal is administered daily for at least 5 months. In some embodiments, the antifungal agent encapsulated in a lipid nanocrystal is administered daily for at least 6 months.

In some embodiments, the antifungal agent encapsulated in a lipid nanocrystal is administered daily for up to 1, 2, 3, 4, 5, 6, 7, 8, 9, 10, 11, or 12 weeks. In some embodiments, the antifungal agent encapsulated in a lipid nanocrystal is administered daily for up to 1 week. In some embodiments, the antifungal agent encapsulated in a lipid nanocrystal is administered daily for up to 2 weeks. In some embodiments, the antifungal agent encapsulated in a lipid nanocrystal is administered daily for up to 3 weeks. In some embodiments, the antifungal agent encapsulated in a lipid nanocrystal is administered daily for up to 4 weeks. In some embodiments, the antifungal agent encapsulated in a lipid nanocrystal is administered daily for up to 5 weeks. In some embodiments, the antifungal agent encapsulated in a lipid nanocrystal is administered daily for up to 6 weeks. In some embodiments, the lipid nanocrystal formulation is administered daily for up to 7 weeks. In some embodiments, the lipid nanocrystal formulation is administered daily for up to 8 weeks. In some embodiments, the lipid nanocrystal formulation is administered daily for up to 9 weeks. In some embodiments, the antifungal agent encapsulated in a lipid nanocrystal is administered daily for up to 10 weeks. In some embodiments, the antifungal agent encapsulated in a lipid nanocrystal is administered daily for up to 11 weeks. In some embodiments, the antifungal agent encapsulated in a lipid nanocrystal is administered daily for up to 12 weeks. In some embodiments, the antifungal agent encapsulated in a lipid nanocrystal is administered daily for up to 3 months. In some embodiments, the antifungal agent encapsulated in a lipid nanocrystal is administered daily for up to 4 months. In some embodiments, the antifungal agent encapsulated in a lipid nanocrystal is administered daily for up to 5 months. In some embodiments, the antifungal agent encapsulated in a lipid nanocrystal is administered daily for up to 6 months.

Where a dosage regimen comprises multiple administrations, it is understood that the effective amount of the lipid nanocrystals or pharmaceutical compositions of the disclosure administered to the subject can comprise the total amount of the lipid nanocrystals or pharmaceutical compositions administered over the entire dosage regimen. The exact amount will depend on the purpose of the treatment, the subject to be treated, and will be ascertainable by a person skilled in the art using known methods and techniques for determining effective doses. In some embodiments, the amount of the antifungal agent that can be administered includes between about 0.1 μg/kg/day to about 100 mg/kg/day. In some embodiments, the amount of the antifungal agent that can be administered includes between about 1.0 μg/kg/day to about 10 mg/kg/day. In some embodiments, the antifungal agent is administered to the subject in an amount ranging from about 1 mg/kg to about 50 mg/kg, such as from about 1 mg/kg to about 40 mg/kg, from about 1 mg/kg to about 30 mg/kg, from about 1 mg/kg to about 20 mg/kg, from about 1 mg/kg to about 10 mg/kg, or from about 1 mg/kg to about 7.5 mg/kg, of patient body weight, whether, for example, by one or more separate administrations.

In some embodiments, the composition described herein is administered to a subject independent of body weight. In some embodiments, the composition is administered to a subject at a fixed dose independent of other clinical factors such as body weight, age, sex, clinical status, etc. In some embodiments, the antifungal agent is administered at a dose of 100-2000 mg/day. In some embodiments, the antifungal agent is administered at a dose of 500-2000 mg/day. In some embodiments, the antifungal agent is administered at a dose of 1000-2000 mg/day. In some embodiments, the antifungal agent is administered at a dose of about 100 mg/day. In some embodiments, the antifungal agent is administered at a dose of about 500 mg/day. In some embodiments, the antifungal agent is administered at a dose of about 1000 mg/day. In some embodiments, the antifungal agent is administered at a dose of about 2000 mg/day.

In some embodiments, the antifungal agent encapsulated in a lipid nanocrystal is formulated as an about 5 mg/mL to about 30 mg/mL suspension. In some embodiments, the antifungal agent encapsulated in a lipid nanocrystal is formulated as a 5 mg/mL suspension. In some embodiments, the antifungal agent encapsulated in a lipid nanocrystal is formulated as a 6 mg/mL suspension. In some embodiments, the antifungal agent encapsulated in a lipid nanocrystal is formulated as a 7 mg/mL suspension. In some embodiments, the antifungal agent encapsulated in a lipid nanocrystal is formulated as a 8 mg/mL suspension. In some embodiments, the antifungal agent encapsulated in a lipid nanocrystal is formulated as a 9 mg/mL suspension. In some embodiments, the antifungal agent encapsulated in a lipid nanocrystal is formulated as a 10 mg/ml suspension. In some embodiments, the antifungal agent encapsulated in a lipid nanocrystal is formulated as a 11 mg/mL suspension. In some embodiments, the antifungal agent encapsulated in a lipid nanocrystal is formulated as a 12 mg/mL suspension. In some embodiments, the antifungal agent encapsulated in a lipid nanocrystal is formulated as a 13 mg/mL suspension. In some embodiments, the antifungal agent encapsulated in a lipid nanocrystal is formulated as a 14 mg/mL suspension. some embodiments, the antifungal agent encapsulated in a lipid nanocrystal is formulated as a 15 mg/mL suspension. In some embodiments, the antifungal agent encapsulated in a lipid nanocrystal is formulated as a 16 mg/mL suspension. In some embodiments, the antifungal agent encapsulated in a lipid nanocrystal is formulated as a 17 mg/mL suspension. In some embodiments, the antifungal agent encapsulated in a lipid nanocrystal is formulated as a 18 mg/mL suspension. In some embodiments, the antifungal agent encapsulated in a lipid nanocrystal is formulated as a 19 mg/mL suspension. In some embodiments, the antifungal agent encapsulated in a lipid nanocrystal is formulated as a 20 mg/mL suspension. In some embodiments, the antifungal agent encapsulated in a lipid nanocrystal is formulated as a 21 mg/mL suspension. In some embodiments, the antifungal agent encapsulated in a lipid nanocrystal is formulated as a 22 mg/mL suspension. In some embodiments, the antifungal agent encapsulated in a lipid nanocrystal is formulated as a 23 mg/mL suspension. In some embodiments, the antifungal agent encapsulated in a lipid nanocrystal is formulated as a 24 mg/mL suspension. In some embodiments, the antifungal agent encapsulated in a lipid nanocrystal is formulated as a 25 mg/mL suspension. In some embodiments, the antifungal agent encapsulated in a lipid nanocrystal is formulated as a 26 mg/mL suspension. In some embodiments, the antifungal agent encapsulated in a lipid nanocrystal is formulated as a 27 mg/mL suspension. In some embodiments, the antifungal agent encapsulated in a lipid nanocrystal is formulated as a 28 mg/mL suspension. In some embodiments, the antifungal agent encapsulated in a lipid nanocrystal is formulated as a 29 mg/mL suspension. In some embodiments, the antifungal agent encapsulated in a lipid nanocrystal is formulated as a 30 mg/mL suspension. In some embodiments, the antifungal agent encapsulated in a lipid nanocrystal is formulated as a 27.5 mg/mL suspension.

“Minimum inhibitory concentration” or “MIC,” defined as the lowest concentration of an antimicrobial that will inhibit the visible growth of a microorganism. MICs are used by diagnostic laboratories mainly to confirm resistance, but most often as a research tool to determine the in vitro activity of new antimicrobials. MICs are generally presented as MIC₅₀ and MIC₉₀ values, which are defined as the lowest concentration of the antimicrobial at which 50% and 90%, respectively, of the isolates tested within a given microorganism species were inhibited. MICs may be determined by methods well known in the art, such as, for example, those described in Jorgensen et al., Clin. Infect. Dis., 2009, 49 (11): 1749-1755 and Schwarz et al., J. Antimicrob. Chemother., 2010, 65 (4): 601-604.

In some embodiments, the MIC₅₀ (μg/mL) of the lipid nanocrystals comprising the antifungal agent, such as amphotericin B, according to the present disclosure is less than about 0.01, such as about 0.009, 0.008, 0.007, 0.006, 0.005, 0.004, 0.003, 0.002, or 0.001. In some embodiments, the MIC₅₀ (μg/mL) of the lipid nanocrystals comprising the antifungal agent, such as amphotericin B, according to the present disclosure is less than about 0.005. In some embodiments, the MIC₅₀ (μg/mL) of the lipid nanocrystals comprising the antifungal agent, such as amphotericin B, according to the present disclosure is less than about 0.003.

In some embodiments, the MIC₉₀ (μg/mL) of the lipid nanocrystals comprising the antifungal agent, such as amphotericin B, according to the present disclosure is less than about 0.05, such as about 0.04, 0.03, 0.02, or 0.01. In some embodiments, the MIC₉₀ (μg/mL) of the lipid nanocrystals comprising the antifungal agent, such as amphotericin B, according to the present disclosure is less than about 0.03. In some embodiments, the MIC₉₀ (μg/mL) of the lipid nanocrystals comprising the antifungal agent, such as amphotericin B, according to the present disclosure is less than about 0.01.

### Combination Therapy

In some embodiments, the antifungal agent encapsulated in a lipid nanocrystal is administered to a subject in combination with a second antifungal agent. In some embodiments, a subject is administered two or more antifungal agents encapsulated in a lipid nanocrystal. In some embodiments, the two or more antifungal agents are encapsulated in the same lipid nanocrystal. In some embodiments, the two or more antifungal agents are encapsulated in different lipid nanocrystals. In some embodiments, a first antifungal agent is encapsulated in a lipid nanocrystal and one or more additional antifungal agents are not encapsulated in a lipid nanocrystal. In some embodiments, a subject is administered three or more antifungal agents encapsulated in a lipid nanocrystal. In some embodiments, a subject is administered a first antifungal agent encapsulated in a lipid nanocrystal and is subsequently administered a second antifungal agent encapsulated in a lipid nanocrystal.

In some embodiments, the antifungal agent encapsulated in a lipid nanocrystal is administered to a subject receiving a standard of care antifungal therapy. In some embodiments, the antifungal agent encapsulated in a lipid nanocrystal is administered to a subject concurrently with a standard of care antifungal therapy. In some embodiments, the antifungal agent encapsulated in a lipid nanocrystal is administered to a subject sequentially with a standard of care antifungal therapy.

In some embodiments, the antifungal agent encapsulated in a lipid nanocrystal is administered to a subject in combination with fluconazole. In some embodiments, the antifungal agent encapsulated in a lipid nanocrystal is administered to a subject concurrently with fluconazole. In some embodiments, the antifungal agent encapsulated in a lipid nanocrystal is administered to a subject sequentially with fluconazole. In some embodiments, the antifungal agent encapsulated in a lipid nanocrystal is administered to a subject in combination with 5-Flucytosine. In some embodiments, the antifungal agent encapsulated in a lipid nanocrystal is administered to a subject concurrently with 5-Flucytosine. In some embodiments, the antifungal agent encapsulated in a lipid nanocrystal is administered to a subject sequentially with 5-Flucytosine. In some embodiments, the antifungal agent encapsulated in a lipid nanocrystal is administered to a subject in combination with isavuconazole. In some embodiments, the antifungal agent encapsulated in a lipid nanocrystal is administered to a subject concurrently with isavuconazole. In some embodiments, the antifungal agent encapsulated in a lipid nanocrystal is administered to a subject sequentially with isavuconazole. In some embodiments, the antifungal agent encapsulated in a lipid nanocrystal is administered to a subject in combination with posaconazole. In some embodiments, the antifungal agent encapsulated in a lipid nanocrystal is administered to a subject concurrently with posaconazole. In some embodiments, the antifungal agent encapsulated in a lipid nanocrystal is administered to a subject sequentially with posaconazole.

In some embodiments, the lipid nanocrystals described herein comprise two antifungals. In some embodiments, the lipid nanocrystals described herein comprise three antifungals. In some embodiments, the lipid nanocrystals described herein comprise two antifungals selected from amphotericin B, amphotec, posaconazole, isavuconazole, voricoazole, fluconazole, itraconazole, caspofungin acetate, and micafungin.

### Subjects

In some embodiments, a subject is administered a lipid nanocrystal described herein. In some embodiments, the subject is a mammal. In some embodiments, the subject is a human.

In some embodiments, the subject is suspected of having mucormycosis. In some embodiments, the subject is diagnosed with mucormycosis. In some embodiments, the subject is undergoing treatment for mucormycosis.

In some embodiments, the human subject is under the age of about 1 year. In some embodiments, the human subject is over the age of 1 year old.

Major risk factors for contracting mucormycosis include uncontrolled diabetes mellitus that results in hyperglycemia and ketoacidosis (DKA), other forms of acidosis, treatment with corticosteroids, solid organ or bone marrow transplantation, neutropenia, trauma and burns (e.g., wounded soldiers in Iraq and Afghanistan), malignant haematological disorders, and deferoxamine therapy in patients receiving haemodialysis (Ibrahim, A. S. & Kontoyiannis, D. P. Update on mucormycosis pathogenesis. Curr Opin Infect Dis 26, 508-515 (2013); Weintrob, A. C. et al. Combat trauma-associated invasive fungal wound infections: epidemiology and clinical classification. Epidemiol Infect 143, 214-224, doi: 10.1017/S09502688140005 IX (2015); Sugar, A. M. in Principles and Practice of Infectious Diseases Vol. 2 (eds G. L. Mandell, J. E. Bennett, & R. Dolin) 2973-2984 (Elsevier Churchill Livingstone, 2005)).

In some embodiments, the subject is immunosuppressed. In some embodiments, the mucormycosis infection occurs in a subject with diabetes. In some embodiments, mucormycosis infection occurs in a subject with a weakened immune system. In some embodiments, mucormycosis infection occurs in a subject with cancer. In some embodiments, mucormycosis infection occurs in a subject with leukemia. In some embodiments, mucormycosis infection occurs in a subject with lymphoma. In some embodiments, mucormycosis infection occurs in a subject that received a bone marrow transplantation. In some embodiments, mucormycosis infection occurs in a subject the received a solid organ transplant. In some embodiments, mucormycosis infection occurs in a subject with graft-versus-host disease.

In some embodiments, an individual predisposed or at risk of mucormycosis is an individual with AIDS, azotemia, diabetes mellitus, bronchiectasis, emphysema, TB, lymphoma, leukemia, burns, a history of susceptibility to a fungal condition, or any combination thereof. In some embodiments, the subject at risk of infection has uncontrolled diabetes mellitus that results in hyperglycemia and ketoacidosis (DKA), other forms of acidosis, is undergoing treatment with corticosteroids, has undergone solid organ, bone marrow or stem cell transplant, has neutropenia (low number of white blood cells), has a history of injection drug use, elevated iron levels (iron overload or hemochromatosis), skin injury due to surgery, trauma, burns, or wounds (e.g., wounded soldiers), malignant haematological disorders, has undergone deferoxamine therapy and is receiving haemodialysis, or was premature and has low birthweight (for neonatal gastrointestinal mucormycosis).

In some embodiments, the subject is neutropenic. In some embodiments, the subject has diabetic ketoacidosis. In some embodiments, the subject is treated with corticosteroids. In some embodiments, the subject has a burn wound. In some embodiments, the subject has a malignan haematological disorder. In some embodiments, the subject is receiving haemodialysis. In some embodiments, the subject is receiving deferozamine therapy.

### Toxicity

Oral administration of lipid nanocrystal antifungals, such as AmB, exhibit reduced toxicity as compared to parenteral administration of unencochleated antifungals. Subjects treated with parenteral, unencochleated antifungals, such as AmB must be under close clinical observation because of the potential, e.g, for nephrotoxicity, which is associated with their use.

Administering antifungal agents, such as AmB, as part of a lipid nanocrystal formulation reduces the toxicity associated with this antifungal and permits the administration of higher doses of AmB. The lower toxicity for lipid nanocrystal antifungals such as AmB, permits such antifungals to be delivered orally at lower doses with improved efficacy and reduced toxicity. Alternatively, due to the lower toxicity, the lipid nanocrystal antifungals can be administered more frequently and/or at higher doses with less risk of adverse consequences.

### Lipid Nanocrystals and Methods of Making the Same

Lipid nanocrystals (i.e., cochleates) are anhydrous, stable, multi-layered lipid crystals which spontaneously form upon the interaction of negatively charged lipids, such as phosphatidylserine, and divalent cations, such as, calcium (see, for example, U.S. Pat. Nos. 4,078,052; 5,643,574; 5,840,707; 5,994,318; 6,153,217; 6,592,894, as well as PCT Publ. Nos. WO 2004/091572; WO 2004/091578; WO 2005/110361, WO 2012/151517, and WO2014/022414, U.S. Patent Publication No. 2014/220108 and Patent Publication No. 2010/0178325; each of which is incorporated fully herein by this reference). In some embodiments, these are referred to as crystal cochleates.

Lipid nanocrystals have a unique multilayered structure consisting of a large, continuous, solid, phospholipid bilayer sheet or strata rolled up in a spiral or as stacked sheets, with no internal aqueous space. This unique structure provides protection from degradation for associated “encochleated” molecules. Since the entire lipid nanocrystal structure is a series of solid layers, components within the interior of the lipid nanocrystal structure remain intact, even though the outer layers of the lipid nanocrystal may be exposed to harsh environmental conditions or enzymes. Divalent cation concentrations in vivo in serum and mucosal secretions are such that the lipid nanocrystal structure is maintained. Hence, the majority of lipid nanocrystal-associated molecules are present in the inner layers of a solid, stable, impermeable structure. Once within the interior of a cell, however, the low calcium concentration results in the opening of the lipid nanocrystal and release of the molecule that had been formulated into lipid nanocrystals. Accordingly, lipid nanocrystal formulations remain intact in physiological fluids, including mucosal secretions, plasma, and gastrointestinal fluid, thereby mediating the delivery of biologically active compounds by many routes of administration, including mucosal, e.g., oral or intransal administration.

Typical lipid nanocrystal structures include a lipid strata comprising alternating divalent cations and phospholipid bilayers that include at least one negatively charged phospholipid.

In some embodiments, a cargo moiety is sequestered within the lipid strata of the lipid nanocrystal. In some embodiments, an antifungal agent is sequestered within the lipid strata of the lipid nanocrystal. In some embodiments, one or more of amphotericin B (AmB), amphotec, posaconazole, isavuconazole, voricoazole, fluconazole, itraconazole, caspofungin acetate, micafungin, are sequestered within the lipid strata of the lipid nanocrystal. In some embodiments, amphotericin B is sequestered within the lipid strata of the lipid nanocrystal. In some embodiments, amphotec is sequestered within the lipid strata of the lipid nanocrystal. In some embodiments, posaconazole is sequestered within the lipid strata of the lipid nanocrystal. In some embodiments, isavuconazole is sequestered within the lipid strata of the lipid nanocrystal. In some embodiments, voricoazole is sequestered within the lipid strata of the lipid nanocrystal. In some embodiments, fluconazole is sequestered within the lipid strata of the lipid nanocrystal. In some embodiments, itraconazole is sequestered within the lipid strata of the lipid nanocrystal. In some embodiments, caspofungin acetate is sequestered within the lipid strata of the lipid nanocrystal. In some embodiments, micafungin is sequestered within the lipid strata of the lipid nanocrystal.

Lipid nanocrystals can be made using known methods. In some embodiments, the method described in U.S. Patent Publication No. 2014/220108 is used to make the lipid nanocrystal of the present disclosure, which is herein incorporated by reference in its entirety. In this method, a hydrophobic antifungal compound, such as amphotericin B, is dissolved in solvent, e.g., dimethylsulfoxide and filtered through e.g., a 0.22 μm filter and combined with e.g., 2000 milligrams 50% soy phosphatidylserine (PS) liposomes in 200 milliliters sterile water (the PS liposomes are first filtered through e.g., 5, 0.8, and 0.45 μm filters) to form liposomes containing the antifungal, such as AmB. To the resultant mixture a cation, such as a multivalent or divalent cation, can be added. The addition of a multivalent or divalent cation results in the collapse of the liposomes, and the formation of the sheets of cation-chelated phospholipid bilayers, which roll up or stack to form lipid nanocrystals containing antifungal, such as AmB. In some embodiments, antifungal-containing lipid nanocrystals, such as AmB-containing lipid nanocrystals, are dried under lyophilization. In some embodiments, sterile water is added to the dried powder, anti-fungal lipid nanocrystals to prepare a suspension. In some embodiments, the suspension is stored at 4 °C in the absence of light.

Other methods for making lipid nanocrystals containing antifungals include the trapping-high pH method, the trapping-film method and the hydrogel method. In some embodiments, the lipid nanocrystals containing antifungals described herein are made using the trapping-high pH method. In some embodiments, the lipid nanocrystals containing antifungals described herein are made using the trapping-film method. In some embodiments, the lipid nanocrystals containing antifungals described herein are made using the hydrogel method.

In the trapping-high pH method, lipid powder and an antifungal compound, e.g., AmB, are mixed in a lipid/antifungal molar ratio of e.g., 10:1 in an e.g., sterile polypropylene tube. Buffer, e.g., TES [N-Tris(hydroxymethyl)-methyl-2-aminomethane sulfonic acid] (pH 7.4) is added. Multilamellar liposomes are formed after vortexing. The pH is then increased to, e.g., 11.5, by the addition of e.g., 1 N NaOH, to solubilize the antifungal compound, e.g., AmB. The absence of AmB crystals and the presence of liposomes may be monitored by using phase contrast and polarization optical microscopy. Multivalent or divalent cation, such as calcium chloride, is added slowly to the antifungal liposome suspension at a lipid/cation molar ratio of e.g., 2:1, to form the lipid nanocrystals. The external pH may then be adjusted to pH 7.

In the trapping film method, antifungal compound, e.g., AmB, is dissolved in solvent, e.g., methanol, with brief sonication and the solution is added to lipids in chloroform. The antifungal, e.g., AmB, is readily soluble in the chloroform/methanol mixture. The mixture may then be dried to a film using a rotary evaporator and gently warmed at e.g., 35 °C-40 °C, under reduced pressure (1 bar). The dried lipid film may then be hydrated with deionized water and sonicated. The antifungal-liposome size should be around 50 nanometers. To form the lipid nanocrystals, a multivalent or divalent cation solution, e.g., calcium chloride in solution, is slowly added to the liposome suspension to form the lipid nanocrystals.

To prepare antifungal lipid nanocrystals using the hydrogel method, an antifungal compound (e.g., AmB) is dissolved in methanol and added to lipids in chloroform at, e.g., a 10:1 molar ratio, and the mixture is then dried into a drug-lipid film using a rotary evaporator. The film may then be hydrated with deionized water and the drug-lipid suspension sonicated until small liposomes containing the anti-fungal compounds are obtained. The antifungal-liposome suspension may then be mixed with e.g., 40% w/w dextran-500,000 in a suspension of, e.g., 2/1 v/v dextran/liposome. This mixture is then injected using a syringe into e.g., 15% w/w PEG-8000 under magnetic stirring (800-1000 rpm). An aqueous-aqueous emulsion of antifungal liposomes/dextran droplets dispersed in a PEG continuous phase is obtained. A multivalent or divalent cation solution, e.g., calcium chloride in solution, is then added to the emulsion. Stirring is continued to allow for the slow formation of small-sized antifungal lipid nanocrystals, which are sequestered in the dextran droplets. The polymer is then washed by the addition of a washing buffer containing e.g., 1 mM CaCl2 and 150 mM NaCl.

As recognized by an ordinary artisan, many parameters, including pH, salt concentration, agitation method and rate, cation type, concentration, and rate of addition, lipid composition, concentration, and ratio of lipid to other material, etc., affect the formulation, and can be varied in order to optimize the encochleation of a particular material.

In some embodiments, a hydrophilic anti-fungal compound, such as 5-FC or an antifungal compound containing a hydrophilic domain, such as fluconazole, may also be formulated into a lipid nanocrystal. Methods for incorporating such compounds into lipid nanocrystals are well known in the art and are described, for example, in U.S. Patent Publication No. 2014/220108. Without wishing to be bound to any particular theory, it is believed that hydrophilic molecules or large molecules with hydrophilic domains, such as active pharmaceutical ingredients (APIs) of interest including the antifungal compounds of the present disclosure, can be formulated into lipid nanocrystals in an enhanced manner by associating the API with a lipid domain that acts like a “raft”, and which remains intact and imbedded within the lipid nanocrystal matrix. Such lipids include “neutral lipids” as known in the art and described herein.

In some embodiments, the ratio of anti-fungal agent to lipid (wt/wt) ranges from 1:1 to 1:50, or any range in between, such as, 1:2, 1:3, 1:4, 1:6, 1:8, 1:10, 1:12, 1:15, 1:20 and 1:25. In some embodiments, the ratio of anti-fungal agent to lipid (wt/wt) ranges from 1:1 to 1:2.5. In some embodiments, the ratio of anti-fungal agent to lipid (wt/wt) ranges from 1:1 to 1:10. In some embodiments, the ratio of anti-fungal agent to lipid (wt/wt) ranges from 1:1 to 1:20. In some embodiments, the ratio of anti-fungal agent to lipid (wt/wt) ranges from 1:1 to 1:25. In some embodiments, the ratio of anti-fungal agent to lipid (wt/wt) ranges from 1:1 to 1:50. In some embodiments, the ratio of anti-fungal agent to lipid (wt/wt) ranges from 1:2.5 to 1:10. In some embodiments, the ratio of anti-fungal agent to lipid (wt/wt) ranges from 1:2.5 to 1:20. In some embodiments, the ratio of anti-fungal agent to lipid (wt/wt) ranges from 1:2.5 to 1:25. In some embodiments, the ratio of anti-fungal agent to lipid (wt/wt) ranges from 1:2.5 to 1:50.

In some embodiments, the ratio of amphotericin B to lipid (wt/wt) ranges from 1:1 to 1:50, or any range in between, such as, 1:2, 1:3, 1:4, 1:6, 1:8, 1:10, 1:12, 1:15, 1:20 and 1:25. In some embodiments, the ratio of amphotericin B to lipid (wt/wt) ranges from 1:1 to 1:2.5. In some embodiments, the ratio of amphotericin B to lipid (wt/wt) ranges from 1:1 to 1:10. In some embodiments, the ratio of amphotericin B to lipid (wt/wt) ranges from 1:1 to 1:20. In some embodiments, the ratio of amphotericin B to lipid (wt/wt) ranges from 1:1 to 1:25. In some embodiments, the ratio of amphotericin B to lipid (wt/wt) ranges from 1:1 to 1:50. In some embodiments, the ratio of amphotericin B to lipid (wt/wt) ranges from 1:2.5 to 1:10. In some embodiments, the ratio of amphotericin B to lipid (wt/wt) ranges from 1:2.5 to 1:20. In some embodiments, the ratio of amphotericin B to lipid (wt/wt) ranges from 1:2.5 to 1:25. In some embodiments, the ratio of amphotericin B to lipid (wt/wt) ranges from 1:2.5 to 1:50.

In some embodiments, the ratio of anti-fungal agent to lipid (wt/wt) is 1:1. In some embodiments, the ratio of anti-fungal agent to lipid (wt/wt) is 1:2. In some embodiments, the ratio of anti-fungal agent to lipid (wt/wt) is 1:3. In some embodiments, the ratio of anti-fungal agent to lipid (wt/wt) is 1:1. In some embodiments, the ratio of anti-fungal agent to lipid (wt/wt) is 1:4. In some embodiments, the ratio of anti-fungal agent to lipid (wt/wt) is 1:5. In some embodiments, the ratio of anti-fungal agent to lipid (wt/wt) is 1:6. In some embodiments, the ratio of anti-fungal agent to lipid (wt/wt) is 1:7. In some embodiments, the ratio of anti-fungal agent to lipid (wt/wt) is 1:8. In some embodiments, the ratio of anti-fungal agent to lipid (wt/wt) is 1:9. In some embodiments, the ratio of anti-fungal agent to lipid (wt/wt) is 1:10. In some embodiments, the ratio of anti-fungal agent to lipid (wt/wt) is 1:11. In some embodiments, the ratio of anti-fungal agent to lipid (wt/wt) is 1:12. In some embodiments, the ratio of anti-fungal agent to lipid (wt/wt) is 1:13. In some embodiments, the ratio of anti-fungal agent to lipid (wt/wt) is 1:14. In some embodiments, the ratio of anti-fungal agent to lipid (wt/wt) is 1:15. In some embodiments, the ratio of anti-fungal agent to lipid (wt/wt) is 1:16. In some embodiments, the ratio of anti-fungal agent to lipid (wt/wt) is 1:17. In some embodiments, the ratio of anti-fungal agent to lipid (wt/wt) is 1:18. In some embodiments, the ratio of anti-fungal agent to lipid (wt/wt) is 1:19. In some embodiments, the ratio of anti-fungal agent to lipid (wt/wt) is 1:20. In some embodiments, the ratio of anti-fungal agent to lipid (wt/wt) is 1:21. In some embodiments, the ratio of anti-fungal agent to lipid (wt/wt) is 1:22. In some embodiments, the ratio of anti-fungal agent to lipid (wt/wt) is 1:23. In some embodiments, the ratio of anti-fungal agent to lipid (wt/wt) is 1:24. In some embodiments, the ratio of anti-fungal agent to lipid (wt/wt) is 1:25. In some embodiments, the ratio of anti-fungal agent to lipid (wt/wt) is 1:30. In some embodiments, the ratio of anti-fungal agent to lipid (wt/wt) is 1:35. In some embodiments, the ratio of anti-fungal agent to lipid (wt/wt) is 1:40. In some embodiments, the ratio of anti-fungal agent to lipid (wt/wt) is 1:45. In some embodiments, the ratio of anti-fungal agent to lipid (wt/wt) is 1:50.

In some embodiments, the ratio of amphotericin B to lipid (wt/wt) is 1:1. In some embodiments, the ratio of amphotericin B to lipid (wt/wt) is 1:2. In some embodiments, the ratio of amphotericin B to lipid (wt/wt) is 1:3. In some embodiments, the ratio of amphotericin B to lipid (wt/wt) is 1:1. In some embodiments, the ratio of amphotericin B to lipid (wt/wt) is 1:4. In some embodiments, the ratio of amphotericin B to lipid (wt/wt) is 1:5. In some embodiments, the ratio of amphotericin B to lipid (wt/wt) is 1:6. In some embodiments, the ratio of amphotericin B to lipid (wt/wt) is 1:7. In some embodiments, the ratio of amphotericin B to lipid (wt/wt) is 1:8. In some embodiments, the ratio of amphotericin B to lipid (wt/wt) is 1:9. In some embodiments, the ratio of amphotericin B to lipid (wt/wt) is 1:10. In some embodiments, the ratio of amphotericin B to lipid (wt/wt) is 1:11. In some embodiments, the ratio of amphotericin B to lipid (wt/wt) is 1:12. In some embodiments, the ratio of amphotericin B to lipid (wt/wt) is 1:13. In some embodiments, the ratio of amphotericin B to lipid (wt/wt) is 1:14. In some embodiments, the ratio of amphotericin B to lipid (wt/wt) is 1:15. In some embodiments, the ratio of amphotericin B to lipid (wt/wt) is 1:16. In some embodiments, the ratio of amphotericin B to lipid (wt/wt) is 1:17. In some embodiments, the ratio of amphotericin B to lipid (wt/wt) is 1:18. In some embodiments, the ratio of amphotericin B to lipid (wt/wt) is 1:19. In some embodiments, the ratio of amphotericin B to lipid (wt/wt) is 1:20. In some embodiments, the ratio of amphotericin B to lipid (wt/wt) is 1:21. In some embodiments, the ratio of amphotericin B to lipid (wt/wt) is 1:22. In some embodiments, the ratio of amphotericin B to lipid (wt/wt) is 1:23. In some embodiments, the ratio of amphotericin B to lipid (wt/wt) is 1:24. In some embodiments, the ratio of amphotericin B to lipid (wt/wt) is 1:25. In some embodiments, the ratio of amphotericin B to lipid (wt/wt) is 1:30. In some embodiments, the ratio of amphotericin B to lipid (wt/wt) is 1:35. In some embodiments, the ratio of amphotericin B to lipid (wt/wt) is 1:40. In some embodiments, the ratio of amphotericin B to lipid (wt/wt) is 1:45. In some embodiments, the ratio of amphotericin B to lipid (wt/wt) is 1:50.

The liposome used during the formation of the lipid nanocrystals may be multilamellar (MLV) or unilamellar (ULV), including small unilamellar vesicles (SUV). In some embodiments, the liposome used during formation of the lipid nanocrystals is multilamellar vesicles (MLV). In some embodiments, the liposome used during formation of the lipid nanocrystals is unilamellar vesicles (ULV). In some embodiments, the liposome used during formation of the lipid nanocrystals is small unilamellar vesicles (SUV).

In some embodiments, the concentration of lipid in these liposomal solutions is from about 0.1 mg/mL to 500 mg/mL. In some embodiments, the concentration of lipid in the liposomal solutions is from about 0.5 mg/mL to about 50 mg/mL. In some embodiments, the concentration of lipid in the liposomal solutions is from about 1 mg/mL to about 25 mg/mL. In some embodiments, the concentration of lipid in the liposomal solution is about 0.1 mg/mL. In some embodiments, the concentration of lipid in the liposomal solution is about 1 mg/mL. In some embodiments, the concentration of lipid in the liposomal solution is about 5 mg/mL. In some embodiments, the concentration of lipid in the liposomal solution is about 10 mg/mL. In some embodiments, the concentration of lipid in the liposomal solution is about 15 mg/mL. In some embodiments, the concentration of lipid in the liposomal solution is about 20 mg/mL. In some embodiments, the concentration of lipid in the liposomal solution is about 25 mg/mL. In some embodiments, the concentration of lipid in the liposomal solution is about 30 mg/mL. In some embodiments, the concentration of lipid in the liposomal solution is about 35 mg/mL. In some embodiments, the concentration of lipid in the liposomal solution is about 40 mg/mL. In some embodiments, the concentration of lipid in the liposomal solution is about 45 mg/mL. In some embodiments, the concentration of lipid in the liposomal solution is about 50 mg/mL. In some embodiments, the concentration of lipid in the liposomal solution is about 75 mg/mL. In some embodiments, the concentration of lipid in the liposomal solution is about 100 mg/mL. In some embodiments, the concentration of lipid in the liposomal solution is about 150 mg/mL. In some embodiments, the concentration of lipid in the liposomal solution is about 200 mg/mL. In some embodiments, the concentration of lipid in the liposomal solution is about 250 mg/mL. In some embodiments, the concentration of lipid in the liposomal solution is about 300 mg/mL. In some embodiments, the concentration of lipid in the liposomal solution is about 350 mg/mL. In some embodiments, the concentration of lipid in the liposomal solution is about 400 mg/mL. In some embodiments, the concentration of lipid in the liposomal solution is about 450 mg/mL. In some embodiments, the concentration of lipid in the liposomal solution is about 500 mg/mL.

A size-regulating agent may be introduced during the method of making the lipid nanocrystal. A size-regulating agent, as used herein, refers to an agent that reduces the particle size of a lipid nanocrystal. As used herein, the term “particle size” refers to the particle diameter, or in case the particles are not spherical, to the largest extension in one direction of the particle. The particle size of lipid nanocrystals can be measured using conventional methods, such as a submicron particle size analyzer. In some embodiments, the size regulating agent is a lipid-anchored polynucleotide, a lipid-anchored sugar (glycolipid), or a lipid-anchored polypeptide. In some embodiments, the size regulating agent is a bile salt, such as oxycholate, cholate, chenodeoxycholate, taurocholate, glycocholate, taurochenodeoxycholate, glycochenodeoxycholate, deoxycholate, or lithocholate. Bile salts are bile acids compounded with a cation, usually sodium. Bile acids are steroid acids found predominantly in the bile of mammals and are commercially available.

In some embodiments, the size-regulating agent is added to the lipid or liposomes before formation of the precipitated lipid nanocrystal. For example, in some embodiments, the size-regulating agent is introduced into a liposomal suspension from which lipid nanocrystals will subsequently be formed (e.g., by addition of cation or dialysis). Alternatively, the size-regulating agent may be introduced to a lipid solution, before or after addition of a pharmacologically active agent.

In some embodiments, the lipid nanocrystals of the present invention can optionally include one or more aggregation inhibitors. The term “aggregation inhibitor,” as used herein, refers to an agent that inhibits aggregation of lipid nanocrystals. The aggregation inhibitor typically is present at least on the surface of the lipid nanocrystal, and may only be present on the surface of the lipid nanocrystal (e.g., when the aggregation inhibitor is introduced after lipid nanocrystal formation). Aggregation inhibitors can be added before, after, or during lipid nanocrystal formation. In some embodiments, an aggregation inhibitor is added before lipid nanocrystal formation. In some embodiments, an aggregation inhibitor is added after lipid nanocrystal formation. In some embodiments, an aggregation inhibitor is added during lipid nanocrystal formation. A person of ordinary skill in the art will readily be able to determine the amount of aggregation inhibitor needed to form lipid nanocrystals of the desired size with no more than routine experimentation.

Suitable aggregation inhibitors that can be used in accordance with the present disclosure, include but are not limited to at least one of the following: polysorbate (e.g., TWEEN), casein, kappa-casein, milk, albumin, serum albumin, bovine serum albumin, rabbit serum albumin, methylcellulose, ethylcellulose, propylcellulose, hydroxycellulose, hydroxymethyl cellulose, hydroxyethyl cellulose, hydroxypropyl cellulose, hydroxypropylmethyl cellulose, polyvinyl pyrrolidone, carboxymethyl cellulose, carboxyethyl cellulose, pullulan, polyvinyl alcohol, sodium alginate, polyethylene glycol, polyethylene oxide, xanthan gum, tragacanth gum, guar gum, acacia gum, arabic gum, polyacrylic acid, methylmethacrylate copolymer, carboxy polymer, amylose, high amylose starch, hydroxypropylated high amylose starch, dextrin, pectin, chitin, chitosan, levan, elsinan, collagen, gelatin, zein, gluten, carrageenan, carnauba wax, shellac, latex polymers, milk protein isolate, soy protein isolate, whey protein isolate and mixtures thereof.

Any suitable lipid can be used to make the lipid nanocrystal. In some embodiments, the lipid includes one or more negatively charged lipids. As used herein, the term “negatively charged lipid” includes lipids having a head group bearing a formal negative charge in aqueous solution at an acidic, basic or physiological pH, and also includes lipids having a zwitterionic head group. In some embodiments, the negatively charged lipid is a phospholipid. The negatively charged phospholipid can be natural, such as soy-based, or synthetic. Examples of negatively charged phospholipids can include, but are not limited to, phosphatidylserine (PS), dioleoylphosphatidylserine (DOPS), phosphatidic acid (PA), phosphatidylinositol (PI), and phosphatidylglycerol (PG). In some embodiments, the negatively charged phospholipid is phosphatidylserine (PS). In some embodiments, the negatively charged phospholipid is dioleoylphosphatidylserine (DOPS). In some embodiments, the negatively charged phospholipid is phosphatidic acid (PA). In some embodiments, the negatively charged phospholipid is phosphatidylinositol (PI). In some embodiments, the negatively charged phospholipid is phosphatidylglycerol (PG). In some embodiments, the lipid component of the lipid nanocrystals according to the disclosure comprises a natural, such as soy-based, negatively charged phospholipid. In some embodiments, the lipid component of the lipid nanocrystals comprises a synthetic negatively charged phospholipid. In some embodiments, the lipid component of the lipid nanocrystals comprises soy-based PS (or “Soy PS”). In some embodiments, the lipid component of the lipid nanocrystals comprises synthetic PS.

The amount of the negatively charged phospholipid in the lipid component of the lipid nanocrystals disclosed herein, such as PS (e.g., Soy PS), can be any amount between from about 50% to about 100%, such as from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 50% to about 90%, from about 60% to about 80%, from about 70% to about 90%, or from about 75% to about 90%, by weight of the lipid component. In some embodiments, the amount of the negatively charged phospholipid in the lipid component of the lipid nanocrystals disclosed herein, such as PS (e.g., Soy PS), is about 50%, about 55%, about 60%, about 65%, about 70%, about 75%, about 80%, about 85%, about 90%, about 95%, or about 100% by weight of the lipid component. In some embodiments, the amount of negatively charged phospholipid in the lipid component of the lipid nanocrystals is about 50%. In some embodiments, the amount of negatively charged phospholipid in the lipid component of the lipid nanocrystals is about 60%. In some embodiments, the amount of negatively charged phospholipid in the lipid component of the lipid nanocrystals is about 70%. In some embodiments, the amount of negatively charged phospholipid in the lipid component of the lipid nanocrystals is about 80%. In some embodiments, the amount of negatively charged phospholipid in the lipid component of the lipid nanocrystals is about 90%. In some embodiments, the amount of negatively charged phospholipid in the lipid component of the lipid nanocrystals is about 100%. All ranges and values between 50% and 100% by weight of the lipid component for the negatively charged phospholipid are meant to be encompassed herein.

In some embodiments, the lipid component of the lipid nanocrystals according to the disclosure can also include non-negatively charged lipids (e.g., positive and/or neutral lipids). In some embodiments, a majority of the lipid component is negatively charged. When the lipid component of the lipid nanocrystals disclosed herein comprises less than about 100% negatively charged phospholipid, such as PS (e.g., Soy PS), the lipid component can further comprise a non-negatively charged lipids, such as a neutrally charged lipid, in an amount no more than about 50%, such as about 45%, about 40%, about 35%, about 30%, about 25%, about 20%, about 15%, about 10%, about 5%, or less than about 5%, by weight of the lipid component. In some embodiments, when the lipid component of the lipid nanocrystals disclosed herein comprises less than about 100% negatively charged phospholipid, such as PS (e.g., Soy PS), the lipid component further comprises a non-negatively charged lipid, such as a neutrally charged lipid, in an amount no more than about 50%, such as about 45%, about 40%, about 35%, about 30%, about 25%, about 20%, about 15%, about 10%, about 5%, or less than about 5%, by weight of the lipid component. The non-negatively charged lipid can be natural, such as soy-based, or synthetic. Examples of such non-negatively charged phospholipids can include, but are not limited to, phosphatidylcholine (PC), phosphatidylethanolamine (PE), diphosphotidylglycerol (DPG), dioleoyl phosphatidic acid (DOPA), distearoyl phosphatidylserine (DSPS), dimyristoyl phosphatidylserine (DMPS), dipalmitoyl phosphatidylgycerol (DPPG) and the like. In some embodiments, the lipid component of the lipid nanocrystals according to the disclosure comprises, in addition to a negatively charged phospholipid, such as PS (e.g., Soy PS), a neutrally charged lipid, such as PC. In some embodiments, the lipid component of the lipid nanocrystals according to the disclosure comprises, in addition to a negatively charged phospholipid the neutrally charged lipid phosphatidylcholine (PC). In some embodiments, the lipid component of the lipid nanocrystals according to the disclosure comprises, in addition to a negatively charged phospholipid the neutrally charged lipid phosphatidylethanolamine (PE). In some embodiments, the lipid component of the lipid nanocrystals according to the disclosure comprises, in addition to a negatively charged phospholipid the neutrally charged lipid diphosphotidylglycerol (DPG). In some embodiments, the lipid component of the lipid nanocrystals according to the disclosure comprises, in addition to a negatively charged phospholipid the neutrally charged lipid dioleoyl phosphatidic acid (DOPA). In some embodiments, the lipid component of the lipid nanocrystals according to the disclosure comprises, in addition to a negatively charged phospholipid the neutrally charged lipid distearoyl phosphatidylserine (DSPS). In some embodiments, the lipid component of the lipid nanocrystals according to the disclosure comprises, in addition to a negatively charged phospholipid the neutrally charged lipid dimyristoyl phosphatidylserine (DMPS). In some embodiments, the lipid component of the lipid nanocrystals according to the disclosure comprises, in addition to a negatively charged phospholipid the neutrally charged lipid dipalmitoyl phosphatidylgycerol (DPPG).

In some embodiments, the lipid chains of the phospholipids are from about 6 to about 26 carbon atoms, and the lipid chains can be saturated or unsaturated. In some embodiments, the lipid chains are 6, 7, 8, 9, 10, 11, 12, 13, 14, 15, 16, 17, 18, 19, 20, 21, 22, 23, 24, 25, or 26 carbon atoms in length. In some embodiments, the lipid chains are saturated. In some embodiments, the lipid chains are unsaturated. Fatty acyl lipid chains useful in the present disclosure include, but are not limited to, n-tetradecanoic, n-hexadecanoic acid, n-octadecanoic acid, n-eicosanoic acid, n-docosanoic acid, n-tetracosanoic acid, n-hexacosanoic acid, cis-9-hexadecenoic acid, cis-9-octadecenoic acid, cis, cis-9,12-octadecedienoic acid, all-cis-9,12,15-octadecetrienoic acid, all-cis-5,8,11,14-eicosatetraenoic acid, all-cis-4,7,10,13,16,19-docosahexaenoic acid, 2,4,6,8-tetramethyl decanoic acid, and lactobacillic acid, and the like. In some embodiments, the fatty acyl lipid chain is n-tetradecanoic. In some embodiments, the fatty acyl lipid chain is n-hexadecanoic acid. In some embodiments, the fatty acyl lipid chain is n-octadecanoic acid. In some embodiments, the fatty acyl lipid chain is n-eicosanoic acid. In some embodiments, the fatty acyl lipid chain is n-docosanoic acid. In some embodiments, the fatty acyl lipid chain is n-tetracosanoic acid. In some embodiments, the fatty acyl lipid chain is n-hexacosanoic acid. In some embodiments, the fatty acyl lipid chain is cis-9-hexadecenoic acid. In some embodiments, the fatty acyl lipid chain is cis-9-octadecenoic acid. In some embodiments, the fatty acyl lipid chain is cis, cis-9,12-octadecedienoic acid. In some embodiments, the fatty acyl lipid chain is all-cis-9,12,15-octadecetrienoic acid. In some embodiments, the fatty acyl lipid chain is all-cis-5,8,11,14-eicosatetraenoic acid. In some embodiments, the fatty acyl lipid chain is all-cis-4,7,10,13,16,19-docosahexaenoic acid. In some embodiments, the fatty acyl lipid chain is 2,4,6,8-tetramethyl decanoic acid. In some embodiments, the fatty acyl lipid chain is lactobacillic acid.

In some embodiments, the lipid component of the lipid nanocrystals disclosed herein comprises pharmaceutical grade lipids, such as phospholipids, such as soy phospholipids, such as soy phosphatidylserine. As used herein, “pharmaceutical grade” refers to components, such as phospholipids, that are under strict quality and purity testing, and do not contain binders, fillers, dyes, excipients, or unknown substances.

In other embodiments, the lipid component comprises nutraceutical grade lipids, such as phospholipids, such as soy phospholipids, such as soy phosphatidylserine. As used herein, “nutraceutical grade” refers to a classification introduced in 1990 by the Food and Nutrition Board of the United States Institute of Medicine to describe functional food products that offer medical and/or health benefits.

Pharmaceutical grade and nutraceutical grade lipids comprising about 40% to about 74% phosphatidylserine, such as about 40% to about 74% soy phosphatidylserine, are commercially available from e.g., Chemi Nutra, American Lecithin Company or Lipoid LLC, e.g. LIPOID® PS 70, LIPOID® PS 50 or ALCOLEC®PS 50 P.

In general, the liposome solution used for preparing the lipid nanocrystals of the disclosure has a lipid concentration of from about 0.5 mg/mL to about 100 mg/mL, such as from about 0.75 mg/mL to about 95 mg/mL, from about 1 mg/mL to about 90 mg/mL, from about 1.5 mg/mL to about 85 mg/mL, from about 2 mg/mL to about 80 mg/mL, from about 3 mg/mL to about 60 mg/mL, from about 4 mg/mL to about 50 mg/mL, from about 1 mg/mL to about 30 mg/mL, from about 1 mg/mL to about 20 mg/mL, or from about 0.5 mg/mL to about 10 mg/mL. In some embodiments, the liposome solution has a lipid concentration of about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL.

Typically, a multivalent compound is used to precipitate the lipid nanocrystals of the disclosure from a liposome solution as described herein elsewhere. In some embodiments, the multivalent cation described herein, which may be used to collapse the liposomes into lipid nanocrystals, is a divalent metal cation, such as calcium (Ca⁺⁺), zinc (Zn⁺⁺), magnesium (Mg⁺⁺), and barium (Ba⁺⁺). In some embodiments, the divalent metal cation is calcium (Ca⁺⁺). In some embodiments, the divalent metal cation is (Zn⁺⁺). In some embodiments, the divalent metal cation is magnesium (Mg⁺⁺). In some embodiments, the divalent metal cation is barium (Ba⁺⁺).

The amount of multivalent cation, such as Ca⁺⁺, added to the liposome solution should be in an amount sufficient to precipitate the lipid nanocrystals and generally is expressed in term of the molar ratio between the multivalent cation added and the negatively charged phospholipid comprised in the liposome solution. In some embodiments, the molar ratio of the multivalent cation, such as Ca⁺⁺, added to the liposome solution and the negatively charged phospholipid is from about 0.25:1 to about 4:1, such as from about 0.5:1 to about 4%, from about 1:1 to about 4:1, from about 1.5:1 to about 4:1, from about 2:1 to about 4:1, from about 2.5:1 to about 4:1, from about 3:1 to about 4:1, from about 0.5:1 to about 2:1, or from about 1:1 to about 2.5:1. In some embodiments, the molar ratio of the multivalent cation, such as Ca⁺⁺, added to the liposome solution and the negatively charged phospholipid is about 0.25:1, about 0.5:1, about 0.75:1, about 0.8:1, about 1:1, about 1.25:1, about 1.5:1, about 1.75:1, about 2:1, about 2.25:1, about 2.5:1, about 2.75:1, about 3:1, about 3.25:1, about 3.5:1, about 3.75:1, or about 4:1.

The amount of multivalent cation, such as Ca⁺⁺, added to the liposome solution can also be expressed in term of its final concentration in the population of lipid nanocrystals. Thus, in some embodiments, the population of lipid nanocrystals has a concentration of the multivalent cation, such as Ca⁺⁺, of from about 1 mM to about 12 mM, such as from about 1.5 mM to about 10 mM, from about 2 mM to about 10 mM, from about 2.5 mM to about 8 mM, from about 3 mM to about 6 mM, or from about 3.5 mM to about 6 mM. In some embodiments, the population of lipid nanocrystals has a concentration of the multivalent cation, such as Ca⁺⁺, of about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM, about 10.5 mM, about 11 mM, about 11.5 mM, or about 12 mM.

The lipid nanocrystals of the disclosure can be made by any process known in the art, such as those described in U.S. Pat. Nos. 4,078,052; 5,643,574; 5,840,707; 5,994,318; 6,153,217; 6,592,894, as well as PCT Publ. Nos. WO 2004/091572; WO 2004/091578; WO 2005/110361, WO 2012/151517, and WO2014/022414, and U.S. Pat. Publ. 2010/0178325; each of which is incorporated fully herein by reference. For instance, the lipid nanocrystals of the disclosure can be made by preparing a solution of liposomes comprising the negatively charged phospholipid as described herein in an aqueous medium, combining the solution of liposomes with an antifungal agent, and adding a multivalent cation to precipitate the lipid nanocrystals. In the embodiments where a neutrally charged lipid, such as phosphatidylcholine, is also used to preparing the lipid nanocrystals of the disclosure, the neutrally charged lipid, such as phosphatidylcholine, is added into the solution of liposomes prior to combining the solution of liposomes with the antifungal agent.

The solution of liposomes can be prepared by stirring the lipids comprising the phospholipids and a chelating agent, such as ethylenediaminetetraacetic acid (EDTA) in purified water, thereby forming the liposomes. After formation, the liposomes are typically stirred for about 1, 2, 3 or 4 or more hours, more typically about 4 hours, e.g., at room temperature. In some embodiments, the liposomes are homogenized or filtered after stirring at room temperature, as disclosed in, for instance, US 2021/0038722, which is herein incorporated by reference in its entirety. In some embodiments, the liposomes may be homogenized by passing the liposomes through a homogenizer, such as a PandaPlus 2000 homogenizer (GEA Inc.). In other embodiments, the liposomes are filtered through a 5 μm pre-rinsed filter, such as a syringe 5 μm filter, e.g., obtained from Fisher Scientific, code #SLSVO25LS to remove any insoluble material and produce a more uniform population of liposomes. The liposomes prepared from pharmaceutical grade phospholipids, such as soy phosphatidylserine, are typically filtered twice or homogenized twice and then mixed with water, e.g., purified water to form a liposomal suspension.

### Lipid Nanocrystals Containing Soy Lipids

In some embodiments, the lipid nanocrystals described herein, are prepared using legume-based phospholipids, more typically soy-based lipids. Such soy-based lipids can be natural or synthetic. In some embodiments, the soy-based lipids are soy phospholipids, such as soy phosphatidylserine. In some embodiments, soy phosphatidylserine is in an amount of 40%-74% by weight of the lipid component of the lipid nanocrystals. In some embodiments, the soy phosphatidylserine can be about 40%, 45%, 50%, 55%, 60%, 65% or 70% or any incremental value thereof, by weight of the lipid component of the lipid nanocrystals. It is to be understood that all values, and ranges between these values and ranges are meant to be encompassed by the present disclosure. In some embodiments, the phospholipid comprises 45-70% soy phosphatidylserine. In some embodiments, the phospholipid comprises 45-55% soy phosphatidylserine. In some embodiments, the phospholipid comprises 50%-100% soy phosphatidylserine. In some embodiments, the phospholipid comprises 50% soy phosphatidylserine. In some embodiments, the phospholipid comprises 75% soy phosphatidylserine. In some embodiments, the phospholipid comprises 100% soy phosphatidylserine.

In some embodiments, the soy phosphatidylserine is commercially available, e.g., from Avanti Polar Lipids, Inc. Alabaster, AL. In some embodiments, soy phosphtidylserine is purified from soy phospholipid compositions, which are mixtures of several soy phospholipids, according to well-known and standard purification techniques.

In some embodiments, neutral lipids are used in combination with the soy phosphatidylserine to make lipid nanocrystals. As used herein, the term “neutral lipids” include any of a number of lipid species, which exist either in an uncharged or neutral zwitterionic form at physiological pH and, thus, are included within the group of lipids lacking an anionic function. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the lipid nanocrystal compositions described herein is generally guided by consideration of, e.g., lipid nanocrystal size and stability. Lipids having a variety of acyl chain groups of varying chain length and degree of saturation are available or may be isolated or synthesized by well-known techniques. In some embodiments, lipids containing saturated fatty acids with carbon chain lengths in the range of C14 to C22 can be used. In some embodiments, lipids with mono or di-unsaturated fatty acids with carbon chain lengths in the range of C14 to C22 can be used. In some embodiments, lipids with mono or di-unsaturated fatty acids with carbon chain lengths in the range of C8 to C12 can be used. Additionally, lipids having mixtures of saturated and unsaturated fatty acid chains can be used.

In some embodiments, the neutral lipids used in the present disclosure are DOPE, DSPC, DPPC, POPC, or any related phosphatidylcholine. The neutral lipids useful in the present disclosure may also be composed of sphingomyelin, dihydrosphingomyeline, or phospholipids with other head groups, such as serine and inositol.

In some embodiments, 99.9% pure dioleoyl phosphatidylserine, 99.9% pure soy phosphatidylserine, 75% soy phosphatidylserine and 50% soy phosphatidylserine, are used to manufacture lipid nanocrystals. The lipid composition of 99.9% pure phosphatidylserine is typically modified by the addition of neutral lipids, including, but not limited to sphingomyelin and/or phosphatidylcholine. When lower purity phosphatidylserine (e.g., 50% soy phosphatidylserine) is used as a starting material, the lower purity phosphatidylserine can be subjected to extraction steps to remove unwanted impurities, such as, nucleases.

### Antifungals

The term “antifungal agent” or “antifungal,” as used herein, is intended to mean an agent that destroys fungi, or inhibits or prevents fungal growth, viability and/or virulence. Exemplary categories of antifungal agents include, but are not limited to, polyene antifungal agents, azole antifungal agents and echinocandin antifungal agents. Specific examples of polyene antifungal agents include, but are not limited to, amphotericin B deoxycholate, liposomal amphotericin B, amphotericin B lipid complex and amphotec. Specific examples of azole antifungal agents include, but are not limited to, posaconazole, voricoazole, fluconazole and itraconazole. Specific examples of echinocandin antifungal agents include, but are not limited to, caspofungin acetate and micafungin. Numerous other antifungal agents are well known in the art and are included within the meaning of the term as it is used herein. In some embodiments therefore, the antifungal agent comprised in the lipid nanocrystals of the disclosure is selected from amphotericin B, amphotec, posaconazole, isavuconazole, voricoazole, fluconazole, itraconazole, caspofungin acetate, micafungin, or combinations thereof. In other embodiments, the antifungal agent comprised in the lipid nanocrystals of the disclosure is amphotericin B.

In some embodiments, the antifungal is a polyene antifungal agent. In some embodiments, the antifungal is amphotericin B deoxycholate. In some embodiments, the antifungal is liposomal amphotericin B (AmB). In some embodiments, the antifungal is amphotericin B lipid complex and amphotec.

In some embodiments, the antifungal is an azole antifungal agent. In some embodiments, the antifungal is Posaconazole. In some embodiments, the antifungal is voricoazole. In some embodiments, the antifungal is fluconazole. In some embodiments, the antifungal is itraconazole.

In some embodiments, the antifungal is an echinocandin antifungal agent. In some embodiments, the antifungal is caspofungin acetate. In some embodiments, the antifungal is micafungin.

In some embodiments, the antifungal is selected from amphotericin B, amphotec, posaconazole, isavuconazole, voricoazole, fluconazole, itraconazole, caspofungin acetate, micafungin, or combinations thereof. In some embodiments, the antifungal is amphotericin B. In some embodiments, the antifungal is amphotec. In some embodiments, the antifungal is Posaconazole. In some embodiments, the antifungal is isavuconazole. In some embodiments, the antifungal is voricoazole. In some embodiments, the antifungal is fluconazole. In some embodiments, the antifungal is itraconazole. In some embodiments, the antifungal is caspofungin acetate. In some embodiments, the antifungal is micafungin.

### Pharmaceutical Compositions

In some embodiments, the lipid nanocrystals of the disclosure are formulated as a pharmaceutical composition for administration. In such embodiments, the formulated pharmaceutical composition can further comprise a pharmaceutically acceptable carrier or excipient. Suitable pharmaceutically acceptable carrier or excipient includes, but is not limited to, a buffer (e.g., Tris-HCl, acetate, phosphate) of various pH and ionic strength; an additive, such as gelatin to prevent absorption to surfaces; a protease inhibitor; a permeation enhancer; an anti-oxidant (e.g., ascorbic acid, sodium metabisulfite, butylated hydroxyanisole); a stabilizer (e.g., hydroxypropyl cellulose, hydroxypropylmethyl cellulose); a viscosity increasing agent (e.g., carbomer, colloidal silicon dioxide, ethyl cellulose, guar gum); a sweetener (e.g. aspartame, citric acid); a preservative (e.g., sorbate, thimerosal, benzyl alcohol, parabens, such as sodium methylparaben and/or propylparaben); a flow-aid (e.g., colloidal silicon dioxide), a plasticizer (e.g., diethyl phthalate, triethyl citrate); an emulsifier (e.g., carbomer, hydroxypropyl cellulose, sodium lauryl sulfate); a polymer coating (e.g., poloxamers or poloxamines, hypromellose acetate succinate); a coating and film forming agent (e.g., ethyl cellulose, acrylates, polymethacrylates, hypromellose acetate succinate); an adjuvant; a pharmaceutically acceptable carrier for liquid formulations, such as an aqueous (water, alcoholic/aqueous solution, emulsion or suspension, including saline and buffered media) or non-aqueous (e.g., propylene glycol, polyethylene glycol, and injectable organic esters such as ethyl oleate) solution, suspension, emulsion or oil; and a parenteral vehicle (for subcutaneous, intravenous, intraarterial, or intramuscular injection), including but not limited to, sodium chloride solution, Ringer's dextrose, dextrose and sodium chloride, lactated Ringer's and fixed oils.

The choice of carrier in the pharmaceutical composition may be determined in part by the particular method used to administer the composition. Accordingly, there are a variety of suitable formulations. For example, the pharmaceutical composition can contain preservatives. Suitable preservatives may include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition.

In addition, buffering agents in some embodiments are included in the composition. Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

The lipid nanocrystals of the disclosure, or pharmaceutical compositions comprising the same, may be administered to a subject in need thereof via any convenient route of administration, including oral administration. Suitable preparation forms for administering the lipid nanocrystals or pharmaceutical compositions of the disclosure include, but are not limited to, for example, tablets, capsules, soft capsules, granules, powders, suspensions, emulsions, microemulsions, nanoemulsions, unit dosage forms, solutions, and syrups. In some embodiments, the pharmaceutical composition is prepared as tablets, capsules, soft capsules, granules, powders, suspensions, emulsions, microemulsions, nanoemulsions, unit dosage forms, rings, films, suppositories, solutions, creams, syrups, transdermal patches, ointments or gels. In some embodiments, the lipid nanocrystals of the disclosure, or pharmaceutical compositions comprising the same, are administered to the subject orally.

In some embodiments, the pharmaceutical composition comprises a salt, such as NaCl or a bile salt, such as oxycholate, cholate, chenodeoxycholate, taurocholate, glycocholate, taurochenodeoxycholate, glycochenodeoxycholate, deoxycholate or lithocholate. Bile salts are bile acids compounded with a cation, usually sodium. Bile acids are steroid acids found predominantly in the bile of mammals and are commercially available. In some embodiments, the bile salts comprise cholate. In some embodiments, the bile salts comprise deoxycholate. In yet another embodiment, the bile salts comprise cholate and deoxycholate. In some embodiments, the bile salts consist of cholate and deoxycholate.

In some embodiments, the concentration of NaCl is 1 mM to 1M, 1 mM to 0.5M, 1 mM to 0.1M, 1 mM to 50 mM, 10 mM to 100 mM, 10 mM to 50 mM, 0.1M to 1M, 0.1M to 0.5M, or 0.5M to 1M. In some embodiments, the concentration of the bile salts is 1 mM to 100 mM, 1 mM to 50 mM, 1 mM to 25 mM, 1 mM to 10 mM, 1 mM to 5 mM, 0.1 mM to 5 mM, 0.1 mM to 1 mM, or 0.1 mM to 0.5 mM bile salts.

In some embodiments, a pharmaceutical composition of the disclosure comprises: an antifungal agent, a phospholipid, EDTA, water, vitamin E, calcium chloride, methylparaben, propylparaben, sodium hydroxide, dehydrated alcohol, monobasic potassium phosphate, potassium sorbate, acesulfame potassium and optionally flavoring. In some embodiments, a pharmaceutical composition of the disclosure comprises: amphotoricin B, a phospholipid, EDTA, water, vitamin E, calcium chloride, methylparaben, proplyparaben, sodium hydroxide, dehydrated alcohol, monobasic potassium phosphate, potassium sorbate, acesulfame potassium and optionally flavoring. In some embodiments, the pharmaceutical composition comprises methylcellulose

### Kits

In some embodiments, the disclosure provides a kit comprising a lipid nanocrystal herein, or a composition thereof, described herein, and instructions for use. In some embodiments, the disclosure provides a kit comprising an antifungal encapsulated in a lipid nanocrystal and instructions for administering the antifungal encapsulated in a lipid nanocrystal for treating mucormycosis in a subject. In some embodiments, the kit comprises a lipid nanocrystal herein, or a composition thereof, described herein, and a package insert containing instructions for use of the kit and/or any component thereof. In some embodiments, the kit comprises, in a suitable container, a lipid nanocrystal herein, or a composition thereof, described herein, one or more controls, and various buffers, reagents, enzymes and other standard ingredients well known in the art. In some embodiments, the container comprises at least one vial, well, test tube, flask, bottle, syringe, or other container means, into which the lipid nanocrystal herein, or a composition thereof, is placed, and in some instances, suitably aliquoted. In some embodiments where an additional component is provided, the kit contains additional containers into which this component is placed. The kits can also include a means for containing a lipid nanocrystal herein, or a composition thereof, and any other reagent in close confinement for commercial sale. Containers and/or kits can include labeling with instructions for use and/or warnings.

In some embodiments, a kit comprises a lipid nanocrystal herein, or a composition thereof, described herein, and a pharmaceutically acceptable carrier, or a pharmaceutical composition comprising the lipid nanocrystal and instructions for treating or delaying progression of mucormycosis in a subject in need thereof. In some embodiments, a kit comprises a lipid nanocrystal herein, or a composition thereof, described herein, and a pharmaceutically acceptable carrier, or a pharmaceutical composition comprising the lipid nanocrystal and instructions for treating or delaying progression of mucormycosis in lung tissue in a subject in need thereof. In some embodiments, a kit comprises a lipid nanocrystal herein, or a composition thereof, described herein, and a pharmaceutically acceptable carrier, or a pharmaceutical composition comprising the lipid nanocrystal and instructions for treating or delaying progression of mucormycosis in brain tissue in a subject in need thereof.

### OTHER EMBODIMENTS

Embodiment 1. A method of treating or preventing mucormycosis in a subject in need thereof, the method comprising administering to the subject a therapeutically effective amount of a lipid nanocrystal comprising an antifungal agent, wherein the lipid nanocrystal comprises a multivalent cation, the antifungal agent, and a lipid component comprising a negatively charged phospholipid.

Embodiment 2. The method of Embodiment 2, wherein the antifungal agent is in an aqueous medium and encapsulated in the lipid nanocrystal.

Embodiment 3. The method of Embodiment 1 or 2, wherein the antifungal agent is selected from the group consisting of amphotericin B, amphotec, posaconazole, isavuconazole, voricoazole, fluconazole, itraconazole, caspofungin acetate, micafungin, and combinations thereof.

Embodiment 4. The method of any one of Embodiments 1-3, wherein the antifungal agent is amphotericin B.

Embodiment 5. The method of any one of Embodiments 1-4, wherein the negatively charged phospholipid comprises phosphatidylserine.

Embodiment 6. The method of any one of Embodiments 1-5, wherein the lipid component further comprises a neutrally charged lipid.

Embodiment 7. The method of Embodiment 6, wherein the neutrally charged lipid comprises phosphatidylcholine.

Embodiment 8. The method of any one of Embodiments 1-7, wherein the multivalent cation is Ca⁺⁺, Zn⁺⁺, Ba⁺⁺, or Mg⁺⁺.

Embodiment 9. The method of Embodiment 8, wherein the multivalent cation is Ca⁺⁺.

Embodiment 10. The method of any one of Embodiments 1-9, wherein the mucormycosis comprises rhinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis.

Embodiment 11. The method of any one of Embodiments 1-10, wherein the mucormycosis is associated with an infectious agent within the order Mucorales.

Embodiment 12. The method of Embodiment 11, wherein the infectious agent is a Rhizopus species or a Mucor species.

Embodiment 13. The method of Embodiment 11 or 12, wherein the infectious agent is selected from Rhizopus oryzae (Rhizopus arrhizus), Rhizopus delemar, Rhizopus microsporus var. rhizopodiformis, Mucor circinelloides, Absidia corymbifera, Apophysomyces elegans, Rhizomucor pusillus and Cunninghamella spp (Cunninghamellaceae family).

Embodiment 14. The method of any one of Embodiments 1-13, wherein the lipid nanocrystal is administered to the subject orally.

Embodiment 15. The method of any one of Embodiments 1-14, wherein the lipid nanocrystal is administered to the subject once daily, twice daily, three times a day, or four times a day.

Embodiment 16. The method of any one of Embodiments 1-15, wherein the lipid nanocrystal is administered to the subject continuously for at least about 7 days.

Embodiment 17. The method of any one of Embodiments 1-16, wherein the antifungal agent is administered to the subject in an amount ranging from about 1 mg/kg to about 50 mg/kg, optionally about 1 mg/kg to about 7.5 mg/kg.

Embodiment 18. The method of any one of Embodiments 1-17, wherein administering the therapeutically effective amount of the lipid nanocrystal reduces fungal burden in the brain.

Embodiment 19. The method of any one of Embodiments 1-18, wherein the antifungal agent is amphotericin B, and wherein orally administering lipid nanocrystal amphotericin B is at least as effective as intravenously administering liposomal amphotericin B.

Embodiment 20. The method of Embodiment 19, wherein the antifungal agent is administered at a lower dosage than the standard of care liposomal amphotericin B.

Embodiment 21. The method of any one of Embodiments 1-20, wherein the MIC₅₀ (μg/mL) of the lipid nanocrystal comprising the antifungal agent is less than 0.005.

Embodiment 22. The method of any one of Embodiments 1-20, wherein the MIC₉₀ (μg/mL) of the lipid nanocrystal comprising the antifungal agent is less than 0.01.

Embodiment 23. The method of any one of Embodiments 1-22, wherein the subject is immunosuppressed.

Embodiment 24. The method of any one of Embodiments 1-23, wherein the subject is a human.

Embodiment 25. The method of any one of Embodiments 1-24, wherein the lipid nanocrystal is formulated as a pharmaceutical composition.

Embodiment 26. The method of Embodiment 25, wherein the pharmaceutical composition further comprises a pharmaceutically acceptable carrier.

### EXAMPLES

The examples provided below are simply for illustrative purposes. Those of skill in the art will be able to readily determine appropriate methods and equipment in order to produce suitable lipid nanocrystals as described herein.

### Example 1. Efficacy of Encochleated Oral Formulation of Amphotericin B in Treating Murine Mucormycosis

The objectives of this study were to determine and compare the efficacy of oral amphotericin B to the current standard of care of liposomal amphotericin B in a neutropenic mouse model of invasive pulmonary mucormycosis.

### Materials and Methods

Rhizopus delemar and Mucor. circinelloides are considered to the be the primary and secondary cause of infection worldwide leading to invasive pulmonary mucormycosis. Current available treatments include isavuconazole and intravenous amphotericin B administration. However, it is estimated almost 40% of patients die within six weeks of diagnosis with mucormycosis even with treatment (Marty F M, et al. Lancet. Infect. Dis., 16:828-37, 2016). The efficacy of lipid nanocrystal (LNC) oral Amphotericin B (MAT2203) compared to the current standard of care of Liposomal amphotericin B (LAMB) in a neuropenic mouse model of invasive pulmonary mucormycosis was investigated.

The drugs used in this study were encochleated, or lipid nanocrystal, oral formulation of amphotericin B (“MAT2203”; dissolved in 5 mM CaCl₂)), liposomal amphotericin B (“LAMB”; Gilead, dissolved in irrigation water and diluted in 5% dextrose water), cyclophosphamide, cortisone acetate, ceftazidime, and enrofloxacin (Baytril; Bayer). Power SYBR™ green PCR master mix (ThermoFisher) was used for DNA analysis. Outbred ICR males (20 to 25 g) were purchased from Envigo (Indianapolis, IN). Syringes used for treatment were 1CC TB SFTY SYR 25GX5100, Monoject™ ½ mL insulin safety syringes with needle, 29 G×½″ (0.33 mm×1.3 cm).

Susceptibility Testing: In vitro susceptibility of MAT2203 against Rhizopus delemar 99-880 and Mucor circineloides f. jenssenii DI15-131 clinical isolates was evaluated using the Clinical Laboratory and Standards Institute (CLSI) M38-A2 method.

Mice and immunosuppression: ICR mice were immunosuppressed with cyclophosphamide (200 mg/kg) and cortisone acetate (500 mg/kg) on days −2, +3, and +8, relative to infection (Luo et al., Antimicrob. Agents Chemother., 2013, 57:3340-3347). To prevent bacterial super-infection and deaths in the immunosuppressed mice, mice received antibacterial prophylaxis consisting of enrofloxacin at 50 ppm enrofloxacin (Baytril; Bayer) in drinking water on day −3, then switched to daily treatment of ceftazidime (5 mg) starting on day 0 through Day +13 relative to infection.

Mice infection and treatment: Immunosuppressed mice were intratracheally infected with 1.5×10⁴, 1.8×10⁴, or 2.5×10⁵ spores (Rhizopus delemar 99-880) or 2.5×10⁶ (M. circinelloides f. jenssenii DI15-131) (1). Therapy with either oral administration of MAT2203 for 7 days or IV administration of LAMB for 4 days started 16 hours post infection. Endpoints were survival of mice at end of observation (D+21), tissue fungal burden as determined by conidial equivalents using qPCR (D+4) (2), and histopathological examination of target organs. All drug treatments were initiated 16 hours post infection and continue for 4 consecutive days given by intravenous injection for LAMB and for 7 days given by oral gavage for MAT2203.

The experimental design of each of the studies is detailed in the following tables.

TABLE 1
Study 1: Survival of mice infected with Rhizopus delemar 99-880.

| Treatment | | | Dosing | | Fre- |
|---|---|---|---|---|---|
| Groups | #Animals | Drug | Route | Dose | quency |
| 1 | 10 | MAT2203 | PO | 5 mg/kg | qd |
| 2 | 10 | MAT2203 | PO | 15 mg/kg | qd |
| 3 | 10 | MAT2203 | PO | 45 mg/kg | qd |
| 4 | 10 | LAMB | i.v. | 10 mg/kg | qd |
| 5 | 10 | None (Placebo) | | | |
| 6 | 5 | Uninfected | | | |

TABLE 2
Study 2: Survival of mice infected with Rhizopus delemar 99-880.

| Treatment | | | Dosing | | Fre- |
|---|---|---|---|---|---|
| Groups | #Animals | Drug | Route | Dose | quency |
| 1 | 10 | MAT2203 | PO | 7.5 mg/kg | bid |
| 2 | 10 | MAT2203 | PO | 15 mg/kg | bid |
| 3 | 10 | MAT2203 | PO | 15 mg/kg | qd |
| 4 | 10 | LAMB | i.v. | 10 mg/kg | qd |
| 5 | 10 | None (Placebo) | | | |
| 6 | 5 | Uninfected | | | |

TABLE 3
Study 3: Tissue fungal burden and histopathological
examination of mice infected with Rhizopus delemar 99-880.

| Treatment | | | Dosing | | Fre- |
|---|---|---|---|---|---|
| Groups | #Animals | Drug | Route | Dose | quency |
| 1 | 12 | MAT2203 | PO | 5 mg/kg | qd |
| 2 | 12 | MAT2203 | PO | 15 mg/kg | qd |
| 3 | 12 | MAT2203 | PO | 45 mg/kg | qd |
| 4 | 12 | LAMB | i.v. | 10 mg/kg | qd |
| 5 | 12 | None (Placebo) | | | |

TABLE 4
Study 4: Survival of mice infected with M. circinelloides
f. jenssenii DI15-131.

| Treatment | | | Dosing | | Fre- |
|---|---|---|---|---|---|
| Groups | #Animals | Drug | Route | Dose | quency |
| 1 | 10 | MAT2203 | PO | 5 mg/kg | qd |
| 2 | 10 | MAT2203 | PO | 15 mg/kg | qd |
| 3 | 10 | MAT2203 | PO | 45 mg/kg | qd |
| 4 | 10 | LAMB | i.v. | 10 mg/kg | qd |
| 5 | 10 | None | | | |
| | | (Placebo) | | | |
| 6 | 5 | Uninfected | | | |
| 7 | 3 | — | — | — | Inoculum |
| | | | | | mice to |
| | | | | | determine |
| | | | | | the |
| | | | | | infectious |
| | | | | | inoculum |

TABLE 5
Tissue fungal burden and histopathological examination of mice
infected with M. circinelloides f. jenssenii DI15-131.

| | # | | Dosing | | | |
|---|---|---|---|---|---|---|
| Group | Animals | Drug | Route | Dose | Frequency | comments |
| 1 | 12 | MAT2203 | PO | Selected from above | qd | |
| 2 | 12 | LAMB | i.v. | 10 mg/kg | qd | Historical control |
| 3 | 12 | None (Placebo) | | | | No treatment control |
| 4 | 3 | — | — | — | — | Inoculum mice to determine |
| | | | | | | the infectious inoculum |
| Total | 39 | | | | | |

Data Evaluation and Statistical Analysis: Differences in survival studies were analyzed by the non-parametric Log Rank test for overall survival and with Mantel-Cox comparisons for median survival times. All other comparisons were conducted with the non-parametric Mann-Whitney test. P values <0.05 was considered statistically significant.

### Results

i. Susceptibility Testing

The in vitro activity of MAT2203 against two clinical isolates of Mucorales fungi was compared to the activity of LAMB using the (CLSI) M38-A2 method. The minimum inhibitory concentrations that resulted in 50% (MIC₅₀) or 90% (MIC₉₀) inhibition of the growth of the fungal spores when compared to no treatment were determined. As shown in Table 6, MAT2203 at a dosage of 5 mg/kg surprisingly showed 5-10-fold increase in in vitro activity against R. delemar and M. circineloides when compared to LAMB at a dosage of 10 mg/kg.

TABLE 6
Minimum Inhibitory Concentrations

| | R. delemar 99-880 | | M. circineloides DI15-131 | |
|---|---|---|---|---|
| | MIC₅₀ | MIC₉₀ | MIC₅₀ | MIC₉₀ |
| Drug | (μg/mL) | (μg/mL) | (μg/mL) | (μg/mL) |
| LAMB | 0.0156 | 0.0315 | 0.0315 | 0.0625 |
| MAT2203 | 0.0016 | 0.0081 | 0.0039 | 0.0078 |

MIC₅₀ and MIC₉₀ are defined as the drug concentration that causes 50% and 90% reduction in growth, respectively.

As a standard of care treatment for certain fungal infections, LAMB, such as AmBisome®, is typically administered intravenously at an initial dosage of 3 mg/kg/day or higher. In view of the MIC₅₀ and MIC₉₀ values for MAT2203 shown in Table 6, which are significantly lower than the MIC₅₀ and MIC₉₀ values for LAMB, MAT2203 can be administered (orally) at dosages lower than the standard of care LAMB.

ii. The In Vivo Activity of MAT2203 Against R. delemar

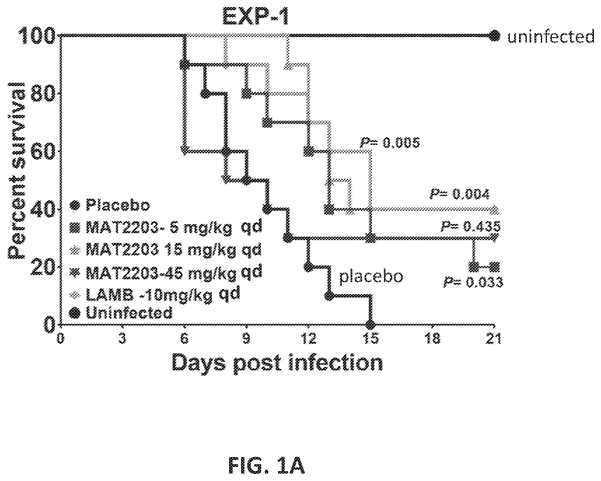

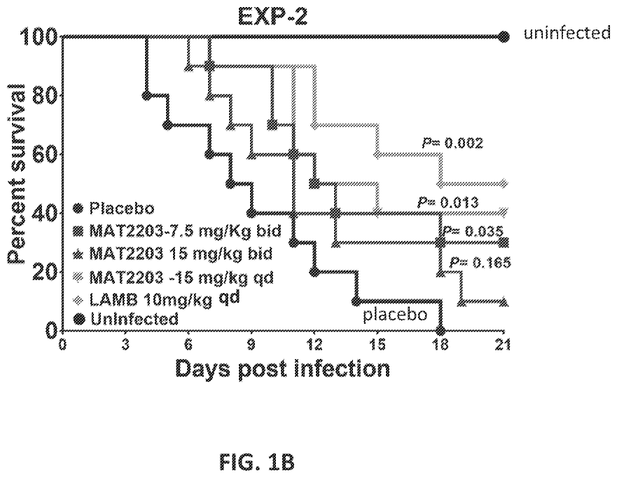

Survival: Two survival experiments were conducted to determine the activity of MAT2203 in treating murine mucormycosis due to R. delemar 99-880. In the first experiment (EXP-1; Table 1), neutropenic mice were infected and treated as detailed above. MAT2203 doses of 5 and 15 mg/kg (qd) showed enhanced overall survival of 20% and 40% when compared to 0% of placebo mice (infection no treatment). A high dose of 45 mg/kg of MAT2203 did not show benefit over placebo treated mice. As expected from previous studies (1, 3), LAMB treatment resulted in a 40% overall survival of mice (FIG. 1A). A repeat study (EXP-2; Table 2) was conducted to confirm the results and to investigate if twice daily treatment with MAT2203 will result in an enhanced benefit in survival of mice infected with R. delemar. MAT2203 at 15 mg/kg given once daily (qd) resulted in a similar protection and an overall survival of neutropenic mice of 40%. In addition, splitting the daily dose of 15 mg/kg into two doses of 7.5 mg/kg/day (bid) resulted in a similar overall survival of 30% when compared to placebo-treated mice of 0% survival. Treating mice with two doses of 15 mg/kg/day did not protect mice from infection. Finally, LAMB treated mice had a historical protection of about 50% (FIG. 1B).

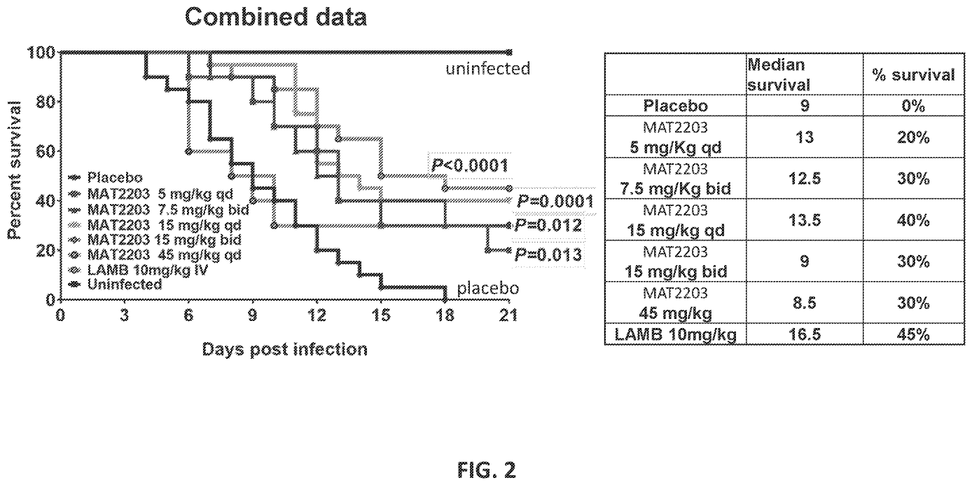

Because EXP-2 results were in agreement with EXP-2, the data were combined and the median survival time of mice in each of the treatment groups was calculated. As can be in seen in FIG. 2, all treatment of MAT2203 resulted in enhanced median survival time (with the exception of the 15 mg/kg bid which had similar median survival time to placebo) and overall survival when compared to placebo-treated mice. Doses of MAT2203 5, 15 mg/kg (qd) or 7.5 mg/kg bid, significantly prolonged median survival time (MST) and enhanced overall survival versus placebo-treated mice (MST of 9 Days and 0% survival for placebo-treated mice versus 13 Days, and 20-40% for MAT2203 doses, P<0.05 by Log-Rank). Importantly, this enhancement in survival was comparable to the current standard of care of LAMB (MST of 16 days and overall survival of 45%).

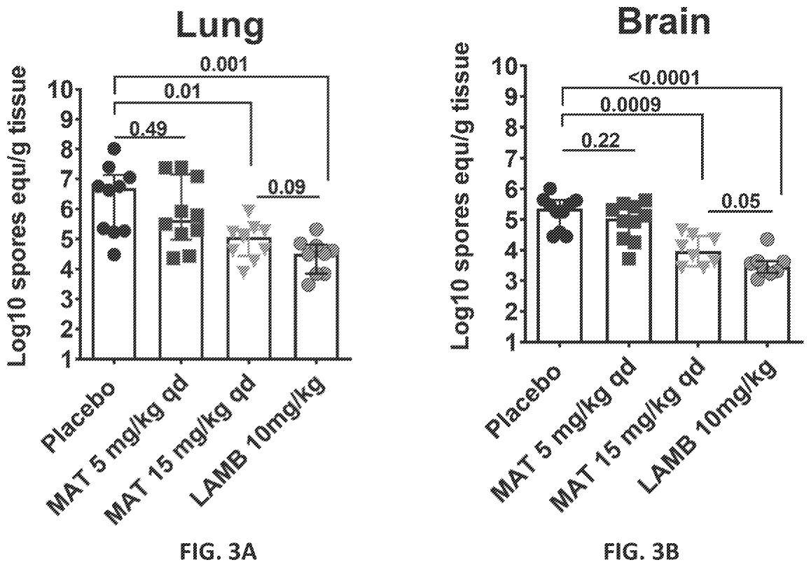

Tissue fungal burden: Because MAT2203 enhanced survival of mice infected with R. delemar, the effect of this drug on the tissue fungal burden of target organs of lung and brain was evaluated (Table 3) (1, 4, 5). Treating mice with 15 mg/kg of MAT2203 once daily resulted in about 1.5 log reduction in lung (FIG. 3A) and 1.0 log reduction in brain fungal spores (FIG. 3B) when compared to placebo-treated mice. Importantly, this reduction in fungal spores was comparable to reduction seen in mice treated with LAMB. While a dose of MAT2203 at 5 mg/kg once daily trended to lower fungal burden in lung, this difference was not significant.

iii. The In Vivo Activity of MAT2203 Against M. circineloides

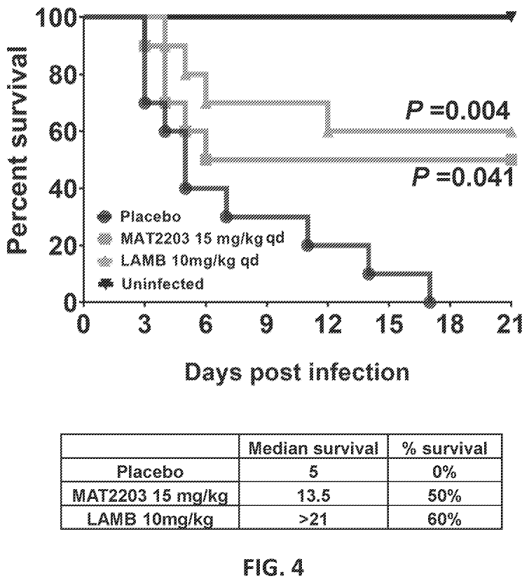

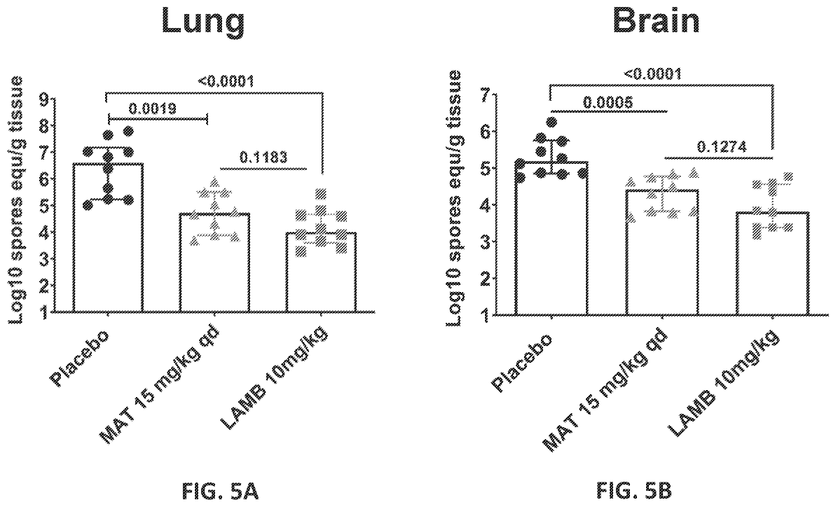

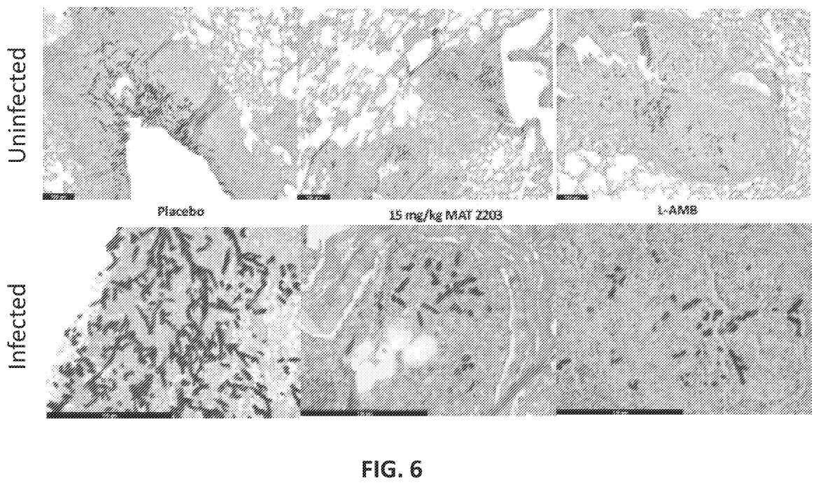

Survival. To investigate if the protective effect of MAT2203 can be expanded to other Mucorales fungi that are commonly isolated from patients with mucormycosis, neutropenic mice were infected with M. circineloides and, 16 hours later, treated with either MAT2203 (15 mg/kg qd) or LAMB (10 mg/kg qd) as detailed above (Table 4). Consistent with the results obtained with mice infected with R. delemar, both drugs prolonged median survival time by 13.5 and >21 days for MAT2203 and LAMB, respectively, when compared to the 5 days of placebo control. Moreover, both drugs enhanced overall 21-day survival to 50% and 60% of MAT2203- and LAMB-treated mice versus 0% survival for placebo control mice (FIG. 4).

Tissue fungal burden and histopathological examination: Because MAT2203 enhanced survival of mice infected with M. circineloides, effect of this drug on the tissue fungal burden of target organs of lung and brain were evaluated (Table 5) (1, 4, 5). Treating mice with 15 mg/kg of MAT2203 once daily resulted in about 2.0 log reduction in lung (FIG. 5A) and about 1.0 log reduction in brain fungal spores when compared to placebo-treated mice (FIG. 5B). Importantly, this reduction in fungal spores was comparable to reduction seen in mice treated with LAMB.

Histopathological examination on the same organs processed for the tissue fungal burden experiment was also conducted. While placebo mice had abscesses full of intact broad aseptate fungal hyphae (consistent with mucormycosis (6)), mice treated with MAT2203 or LAMB had less fungal abscesses with damaged and shorter hyphae (FIG. 6).

Collectively, these results demonstrate the equivalent efficacy of MAT2203 to the current standard of care of LAMB.

### SUMMARY AND CONCLUSIONS

MAT2203 has an in vitro killing activity that is 5-10-fold higher than LAMB against two clinical isolates of R. delemar and M. circinelloides activity. MAT2203 demonstrated an in vivo efficacy in treating R. delemar or M. circinelloides pulmonary infection in immunosuppressed mice. This efficacy is demonstrated by: 1) prolonged median survival time; 2) enhanced overall survival; 3) reduced tissue fungal burden of target organs; and 4) improved histological architecture of infected lungs. The in vivo efficacy of MAT2203 was at least equivalent to the efficacy shown by the current standard of care of LAMB.

### REFERENCES

1. Luo et al., Antimicrob. Agents Chemother., 2013, 57:3340-3347.
2. Ibrahim et al., Antimicrob. Agents Chemother., 2005, 49:721-727.
3. Gebremariam et al., Science Advances, 2019, 5: eaaw1327.
4. Gebremariam et al., J. Clin. Invest., 2014, 124:237-250.
5. Gebremariam et al., J. Clin. Invest., 2016, 126 (6): 2280-2294.
6. Spellberg et al., Clin. Microbiol. Rev., 2005, 18:556-569.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

We claim:

1. A method of treating mucormycosis in a subject in need thereof, the method comprising administering to the subject a composition comprising an antifungal agent encapsulated in a lipid nanocrystal, thereby treating mucormycosis in the subject.

2. A method of preventing mucormycosis in a subject at risk of infection, the method comprising administering to the subject a composition comprising an antifungal agent encapsulated in a lipid nanocrystal, thereby preventing mucormycosis in the subject.

3. The method of claim 1 or claim 2, wherein the mucormycosis is one or more of rhinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis.

4. The method of any one of claims 1-3, wherein the mucormycosis is associated with an infectious agent within the order Mucorales.

5. The method of claim 4, wherein the infectious agent is a Rhizopus species or a Mucor species.

6. The method of claim 4 or 5, wherein the infectious agent is selected from Rhizopus oryzae (Rhizopus arrhizus), Rhizopus delemar, Rhizopus microsporus var. rhizopodiformis, Mucor circinelloides, Absidia corymbifera, Apophysomyces elegans, Rhizomucor pusillus and Cunninghamella spp.

7. The method of any one of claims 1-6, wherein administering comprising oral administration.

8. The method of any one of claims 1-7, wherein the composition is administered to the subject 1-4 times a day for a time period.

9. The method of any one of claims 1-8, wherein the composition is administered once a day for a time period.

10. The method of claim 8 or claim 9, wherein the time period is at least 7 days.

11. The method of any one of claims 1-10, wherein the composition comprises 5-30 mg/mL of the antifungal agent.

12. The method of any one of claims 1-11, wherein 100-2,000 mg/day of the antifungal agent is administered to the subject.

13. The method of any one of claims 1-12, wherein treating comprises reducing fungal burden in the subject.

14. The method of claim 13, wherein fungal burden is reduced in the brain and/or lungs of the subject.

15. The method of claim 13 or 14, wherein the fungal burden is reduced by at least 10%, at least 20%, at least 30%, at least 40%, at least 50%, at least 60%, at least 70%, at least 80%, at least 90%, at least 95%, or at least 99% relative to fungal burden prior to administration of the composition.

16. The method of any one of claims 1-15, wherein the subject is immunosuppressed.

17. The method of any one of claims 1-15, wherein the subject has an HIV infection and/or cancer.

18. The method of any one of claims 1-17 wherein the subject is a human subject.

19. The method of any one of claims 1-18, wherein the antifungal agent is selected from the group: amphotericin B, amphotec, posaconazole, isavuconazole, voricoazole, fluconazole, itraconazole, caspofungin acetate, micafungin, and combinations thereof.

20. The method of any one of claims 1-19, wherein the antifungal agent is amphotericin B.

21. The method of any one of claims 1-20, wherein the lipid nanocrystal comprises a multivalent cation and a lipid component comprising a negatively charged phospholipid.

22. The method of claim 21, wherein the negatively charged phospholipid is phosphatidylserine.

23. The method of claim 22, wherein the phosphatidylserine is soy phosphatidylserine.

24. The method of any one of claims 21-23, wherein the lipid component comprises a neutrally charged lipid.

25. The method of claim 24, wherein the neutrally charged lipid is phosphatidylcholine.

26. The method of any one of claims 21-25, wherein the multivalent cation is Ca⁺⁺, Zn⁺⁺, Ba⁺⁺, or Mg⁺⁺.

27. The method of claim 26, wherein the multivalent cation is Ca⁺⁺.

28. The method of any one of claims 1-27, wherein the MIC₅₀ (μg/mL) of the antifungal agent is less than 0.005.

29. The method of any one of claims 1-27, wherein the MIC₅₀ (μg/mL) of the antifungal agent is less than 0.01.

30. The method of any one of claims 1-29, wherein the composition comprises a pharmaceutically acceptable carrier.

31. The method of any one of claims 1-30, wherein the composition comprises purified water, EDTA, vitamin E, calcium chloride, methylparaben sodium, propylparaben sodium, sodium hydroxide, dehydrated alcohol, monobasic potassium phosphate, potassium sorbate, acesulfame potassium, and optionally flavoring.

32. The method of any one of claims 21-31, wherein the lipid component comprises phosphatidylserine and phosphatidylcholine, and wherein the multivalent cation is Ca⁺⁺.

33. The method of any one of claims 21-31, wherein the lipid component comprises soy phosphatidylserine and phosphatidylcholine, and wherein the multivalent cation is Ca⁺⁺.

34. A method of treating mucormycosis in a subject in need thereof, the method comprising administering to the subject a composition comprising amophotericin B encapsulated in a lipid nanocrystal comprising Ca⁺⁺ and a lipid component comprising phosphatidylserine and phosphatidylcholine, thereby treating mucormycosis in the subject.

35. The method of claim 34, wherein the composition is administered at a dose of 100-2,000 mg amphotericin B per day.

36. Use of a composition for treating mucormycosis in a subject, wherein the composition comprises an antifungal agent encapsulated in a lipid nanocrystal.

37. Use of a composition for preventing mucormycosis in a subject, wherein the composition comprises an antifungal agent encapsulated in a lipid nanocrystal.

38. The use of claim 36 or 37 wherein the mucormycosis is one or more of rhinocerebral mucormycosis, pulmonary mucormycosis, gastrointestinal mucormycosis, disseminated mucormycosis, bone mucormycosis, mediastinum mucormycosis, trachea mucormycosis, kidney mucormycosis, peritoneum mucormycosis, superior vena cava mucormycosis or external otitis mucormycosis.

39. The use of any one of claims 36-38, wherein the mucormycosis is associated with an infectious agent within the order Mucorales, optionally wherein the infectious agent is a Rhizopus species or a Mucor species, further optionally wherein the infectious agent is selected from Rhizopus oryzae (Rhizopus arrhizus), Rhizopus delemar, Rhizopus microsporus var. rhizopodiformis, Mucor circinelloides, Absidia corymbifera, Apophysomyces elegans, Rhizomucor pusillus and Cunninghamella spp.

40. The use of any one of claims 36-39, wherein the composition is formulated for oral administration.

41. The use of any one of claims 36-40, wherein the composition is formulated for administration 1-4 times a day for a time period, optionally wherein the time period is at least 7 days.

42. The use of any one of claims 36-41, wherein the composition comprises 5-30 mg/mL of the antifungal agent.

43. The use of any one of claims 36-42, wherein 100-2,000 mg/day of the antifungal agent is administered to the subject.

44. The use of any one of claims 36-43, wherein the composition reduces fungal burden in the subject, optionally wherein fungal burden is reduced in the brain and/or lungs of the subject, further optionally wherein the fungal burden is reduced by at least 10%, at least 20%, at least 30%, at least 40%, at least 50%, at least 60%, at least 70%, at least 80%, at least 90%, at least 95%, or at least 99% relative to fungal burden prior to administration of the composition.

45. The use of any one of claims 36-44, wherein the subject is immunosuppressed, optionally wherein the subject has an HIV infection and/or cancer.

46. The use of any one of claims 36-45, wherein the antifungal agent is selected from the group: amphotericin B, amphotec, posaconazole, isavuconazole, voricoazole, fluconazole, itraconazole, caspofungin acetate, micafungin, and combinations thereof.

47. The use of any one of claims 36-44, wherein the antifungal agent is amphotericin B.

48. The use of any one of claims 36-47, wherein the lipid nanocrystal comprises a multivalent cation and a lipid component comprising a negatively charged phospholipid.

49. The use of claim 48, wherein the negatively charged phospholipid is phosphatidylserine, optionally wherein the phosphatidylserine is soy phosphatidylserine.

50. The use of any one of claims 48-49, wherein the lipid component comprises a neutrally charged lipid, optionally wherein the neutrally charged lipid is phosphatidylcholine.

51. The use of any one of claims 48-50, wherein the multivalent cation is Ca⁺⁺, Zn⁺⁺, Batt, or Mg⁺⁺, optionally wherein the multivalent cation is Ca⁺⁺.

52. The use of any one of claims 36-51, wherein the MIC₅₀ (μg/mL) of the antifungal agent is less than 0.005 or less than 0.01

53. The use of any one of claims 36-52, wherein the composition comprises a pharmaceutically acceptable carrier.

54. The use of any one of claims 36-53, wherein the composition comprises purified water, EDTA, vitamin E, calcium chloride, methylparaben sodium, propylparaben sodium, sodium hydroxide, dehydrated alcohol, monobasic potassium phosphate, potassium sorbate, acesulfame potassium, and optionally flavoring.

55. A kit comprising a composition comprising an antifungal agent encapsulated in a lipid nanocrystal and instructions for administering the composition to a subject in need thereof, wherein the subject comprises mucormycosis or is at risk of contracting mucormycosis.

56. The kit of claim 55, wherein the antifungal agent is amphotericin B.

57. The kit of claim 55 or 56, wherein the lipid nanocrystal comprises a multivalent cation and a lipid component comprising a negatively charged phospholipid, optionally wherein the multivalent cation is Ca⁺⁺ and the negatively charged phospholipid is phosphatidylserine, further optionally wherein the lipid component comprises a neutrally charged lipid, optionally wherein the neutrally charged lipid is phosphatidylcholine.
